SEQUENCE LISTING (13 sequences)

SEQ ID NO: 1 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 2 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 1458 bp, synthetic construct
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 4 - protein, 21 aa, synthetic construct
MALPVTALLLPLALLLHAARP

SEQ ID NO: 5 - protein, 45 aa, synthetic construct
TTTPAPRPPTPAPTIASQPLSLRPEACRPAAGGAVHTRGLDFACD

SEQ ID NO: 6 - protein, 24 aa, synthetic construct
IYIWAPLAGTCGVLLLSLVITLYC

SEQ ID NO: 7 - protein, 42 aa, synthetic construct
KRGRKKLLYIFKQPFMRPVQTTQEEDGCSCRFPEEEEGGCEL

SEQ ID NO: 8 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 98 aa, synthetic construct
LDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 10 - protein, 39 aa, synthetic construct
IEVMYPPPYLDNEKSNGTIIHVKGKHLCPSPLFPGPSKP

SEQ ID NO: 11 - protein, 27 aa, synthetic construct
FWVLVVVGGVLACYSLLVTVAFIIFWV

SEQ ID NO: 12 - protein, 41 aa, synthetic construct
RSKRSRLLHSDYMNMTPRRPGPTRKHYQPYAPPRDFAAYRS

SEQ ID NO: 13 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2026/0083848 A1
(43) Pub. Date: Mar. 26, 2026

(54) COMBINATION OF CAR T-CELL THERAPY WITH BTK INHIBITORS AND METHODS OF USE THEREOF

(71) Applicant: Viracta Therapeutics, Inc., Cardiff, CA (US)

(72) Inventors: Judith A. FOX, San Francisco, CA (US); Pietro TAVERNA, Watertown, MA (US)

(21) Appl. No.: 18/706,903

(22) PCT Filed: Nov. 2, 2022
(86) PCT No.: PCT/US2022/079166

§ 371 Date: May 2, 2024

Related U.S. Application Data
(60) Provisional application No. 63/275,210, filed on Nov. 3, 2021.

(51) Int. Cl. A61K 40/42; A61K 31/506; A61K 40/11; A61K 40/31; A61P 35/00; A61P 35/02

(52) U.S. Cl. CPC A61K 40/4211; A61K 31/506; A61K 40/11; A61K 40/31; A61P 35/00; A61P 35/02

(57) ABSTRACT

The present disclosure provides the use of immune effector cells (e.g., T-cells or NK cells) engineered to express a Chimeric Antigen Receptor (CAR), in combination with a kinase inhibitor (e.g., a BTK inhibitor), for the treatment of a disease or disorder (e.g., a hematological malignancies).

### RELATED APPLICATIONS

This application claims priority to U.S. Provisional Application No. 63/275,210, filed Nov. 3, 2021, the entire contents of which are incorporated herein by reference.

### SEQUENCE LISTING

The Sequence Listing XML associated with this application is provided electronically in XML format and is hereby incorporated by reference into the specification. The name of the XML file containing the Sequence Listing XML is “SUNS-008_001WO_SeqList”. The XML file is 14,954 bytes in size, created on Oct. 24, 2022, and is being submitted electronically via USPTO Patent Center.

### BACKGROUND

Cell-based therapies (such as adoptive cell therapies) have been shown to be effective for treatment of cancer and other diseases. For example, CD19-targeted chimeric antigen receptor T-cell (CART19) therapy has been successful in treating a subset of patients with hematological malignancies. However, CART19 therapy is associated with significant toxicities, including cytokine release syndrome (CRS), a potentially life-threatening toxicity, and neurotoxicity (NT). Furthermore, the rate of durable responses after CART19 therapy is low, and most patients develop resistance to the therapy.

One of the predominant mechanisms for CART-cell resistance is intrinsic T-cell dysfunction rendering many blood T-cells insufficient to be fit for immunotherapy. Some therapeutics (e.g., bruton's tyrosine kinase (BTK) inhibitor ibrutinib) have been shown to favorably modulate CART phenotype and function through downregulation of inhibitory receptors and enhancement of CART-cell efficacy in preclinical models and early clinical studies. However, ibrutinib leads to drug resistance due to mutations in the BTK C481 kinase domain which is crucial to drug binding.

In contrast to ibrutinib and other BTK inhibitors, vecabrutinib (i.e., (3R,3′R,4′S)-1′-(6-amino-5-fluoropyrimidin-4-yl)-3-((3-chloro-5-(trifluoromethyl) phenyl)amino)-2-oxo-[1,3′-bipiperidine]-4′-carboxamide is a potent, reversible, non-covalent BTK inhibitor that can block the activity of both wild type and C481 mutant kinase.

### SUMMARY OF THE DISCLOSURE

The present disclosure relates to the use of immune effector cells (e.g., T-cells or NK cells) engineered to express a Chimeric Antigen Receptor (CAR), in combination with a kinase inhibitor (e.g., a BTK inhibitor), for the treatment of a disease or disorder (e.g., a hematological malignancy).

In some aspects, the present disclosure provides a combination therapy comprising:
a. immune effector cells (e.g., T-cells or NK cells) engineered to express a Chimeric Antigen Receptor (CAR); and
b. a BTK inhibitor (e.g., vecabrutinib).

In some aspects, the present disclosure provides a combination therapy comprising:
a. CD19 CAR T-cells; and
b. vecabrutinib.

In some aspects, the present disclosure provides a method of treating or preventing a disease or disorder in a subject in need thereof, comprising administering to the subject a therapeutically effective amount of the combination or pharmaceutical composition disclosed herein.

In some aspects, the present disclosure provides a combination or pharmaceutical composition disclosed herein for use in treating or preventing a disease or disorder.

In some aspects, the present disclosure provides a use of the combination disclosed herein in the manufacture of a medicament for treating or preventing a disease or disorder.

In some embodiments, the disease or disorder is a hematological cancer.

In some aspects, the present disclosure provides that a bruton's tyrosine kinase (BTK) inhibitor can ameliorate toxicities after CAR T-cell (CART) therapy for hematological cancers, without significantly impairing anti-tumor effect of the CART therapy.

In some aspects, the present disclosure provides that a BTK inhibitor can ameliorate cytokine release syndrome (CRS) severity or prevent CRS after CAR T-cell therapy for hematological cancers, without significantly impairing anti-tumor effect of the CART therapy.

In some aspects, the present disclosure provides that a BTK inhibitor can ameliorate CRS severity after CAR T-cell therapy for hematological cancers, without significantly impairing anti-tumor effect of the CART therapy.

In some aspects, the present disclosure provides that a BTK inhibitor can prevent CRS after CAR T-cell therapy for hematological cancers, without significantly impairing anti-tumor effect of the CART therapy.

In some aspects, the present disclosure provides that a BTK inhibitor can ameliorate neurotoxicity (NT) or prevent NT after CAR T-cell therapy for hematological cancers, without significantly impairing anti-tumor effect of the CART therapy.

In some aspects, the present disclosure provides that a BTK inhibitor can ameliorate NT after CAR T-cell therapy for hematological cancers, without significantly impairing anti-tumor effect of the CART therapy.

In some aspects, the present disclosure provides that a BTK inhibitor can prevent NT after CAR T-cell therapy for hematological cancers, without significantly impairing anti-tumor effect of the CART therapy.

In some aspects, the present disclosure provides a BTK inhibitor that increases in vivo proliferation of CART cells, following administration of a CART therapy and the BTK inhibitor to a subject in need thereof.

In some aspects, the present disclosure provides a BTK inhibitor that increases in vivo expansion of CART cells, following administration of a CART therapy and the BTK inhibitor to a subject in need thereof.

In some aspects, the present disclosure provides a BTK inhibitor that reduces in vivo immune suppression of CART cells, following administration of a CART therapy and the BTK inhibitor to a subject in need thereof.

In some embodiments, the BTK inhibitor is vecabrutinib.

In some embodiments the CART therapy is CD19 CAR T-cell therapy.

In some embodiments, treating a subject having a disease or disorder described herein, with a combination therapy that includes a CART therapy and a BTK inhibitor (e.g., vecabrutinib) results in improved inhibition or reduction of tumor progression.

In some embodiments, treating a subject having a disease or disorder described herein, with a combination therapy that includes a CART therapy and a BTK inhibitor (e.g., vecabrutinib) results in reduced adverse effects (e.g., reduced CRS) in the subject.

In some embodiments, the treatment effect is as compared to treating a subject with the CART therapy or the BTK inhibitor alone.

In some aspects, the present disclosure provides administering, immune effector cells (e.g., T-cells) expressing a CAR (e.g., a B cell targeting CAR), in combination with a BTK inhibitor (e.g., vecabrutinib).

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the peptides disclosed herein, the chemical structures will control.

Other features and advantages of the disclosure will be apparent from the following detailed description and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A provides a graph quantifying the percentage of CD19-expressing JeKo-1 tumor cells killed following a 24 hour 1:1 in vitro co-culture with primary human T cells transduced with an anti-human CD19 CAR (referred to as CART19 cells) or untransduced (UTD) T cells in the presence of 1 μM vecabrutinib or 10 μM vecabrutinib or a DMSO control.

FIG. 1B provides a graph quantifying the percentage of CD19-expressing JeKo-1 tumor cells killed following a 24 hour in vitro co-culture with CAR19 cells at the indicated effector to target (E:T) ratio in the presence of 1 μM or 10 μM vecabrutinib, 1 μM ibrutinib, or a DMSO control.

FIGS. 2A-2B provide graphs quantifying proliferation of CART19 cells following a 24 hour in vitro co-culture with irradiated JeKo-1 tumor cells at a 1:1 E:T ratio in the presence of DMSO control, 1 μM vecabrutinib or 10 μM vecabrutinib (FIG. 2A) or in the presence of DMSO control or 10 μM ibrutinib (FIG. 2B).

FIG. 3A provides a graph quantifying the percentage of CART19 cells or UTD T cells expressing CD107α (as a marker of degranulation) as measured by flow cytometry following a 4 hour in vitro co-culture with irradiated JeKo-1 tumor cells at a 1:1 E:T ratio in the presence of DMSO control, 1 μM vecabrutinib or 10 μM vecabrutinib.

FIGS. 3B-3D provide graphs quantification of IL-6 (FIG. 3B), IL-10 (FIG. 3C), or MIP-1β (FIG. 3D) in supernatants harvested from a 24 hour co-culture of CART19 cells or UTD T cells with live JeKo-1 cells at a 1:1 E:T ratio in the presence of DMSO control, 1 μM vecabrutinib or 10 μM vecabrutinib.

FIG. 4A provides a graph quantifying bioluminescence as a measure of tumor burden in NSG mice bearing luciferase-expressing JeKo-1 tumors following oral administration of vecabrutinib (50 mg/kg) or vehicle on day −1 and intravenous injection of CART19 cells on day 0. Control mice receive UTD T cells on day 0. Repeat oral administration of vecabrutinib and vehicle was performed twice daily for 4 weeks.

FIG. 4B provides a graph quantifying total number of CD3-expressing CART19 cells as measured by flow cytometry in serum samples obtained on the indicated days from the mice of FIG. 4A.

FIG. 4C provides a graph quantifying percent change in weight of mice of FIG. 4A measured on the indicated days and compared to weight on day 0.

FIG. 5 provides a heat map quantifying fold-change in expression of gene-specific mRNAs harvested from CART19 cells co-cultured with irradiated JeKo-1 cells in the presence of 10 μM vecabrutinib or vehicle (three biological replicates per treatment group).

FIGS. 6A-6C provide plots having a left y-axis quantifying the total level of IP-10 (FIG. 6A), MIP-1β (FIG. 6B), and TNFα (FIG. 6C) in serum obtained from human patients with B cell malignancies either prior to or at 4 weeks following oral administration of vecabrutinib (“pretreatment” and “post-treatment” respectively). The right y-axis depicts the average difference between post-treatment and pretreatment cytokine levels.

### DETAILED DESCRIPTION

The present disclosure relates to the use of immune effector cells (e.g., T-cells or NK cells) engineered to express a Chimeric Antigen Receptor (CAR), in combination with a kinase inhibitor (e.g., a BTK inhibitor), for the treatment of a disease or disorder (e.g., a hematological malignancies). The present disclosure also relates to processes for the preparation of the combination of CART19 and a BTK inhibitor and to their use in the treatment of disorders, such as hematological cancer.

Adoptive immune cell therapies (e.g., adoptive T cell therapy, e.g., CART therapies) have been shown to be effective for treatment of cancer and other diseases. For example, patients having relapsed and refractory chronic lymphocytic leukemia (CLL) and acute lymphocytic leukemia (ALL) have shown durable remissions following administration of autologous T cells genetically modified with a CAR directed to CD19 (see, e.g., Porter, et al. (2011) NEJM 365:725-733; Kaolos, et al (2011) STM 3:95ra73; Grupp, et al (2013) NEJM 368:1509; Maude, et al. (2014) NEJM 371:1507). However, the use of CART therapies for consistently treating a majority of patients is limited in certain disease contexts. For example, complete response rates in CLL are observed in only a fraction of patients (see, e.g., Porter, et al (2015) STM 7:303ra139; Brentjens, et al (2011) Blood 118:4817-28; Kochenderfer, et al (2012) Blood 119:2709-2720).

Moreover, application of CART cell therapy is limited by toxicities that include cytokine release syndrome and neurotoxicity (see, e.g., Neelapu, et al (2017) NEJM 377:2531; Schuster, et al (2017) NEJM 377:2545, Grupp, et al (2013) NEJM 368:1509; Porter, et al (2011) NEJM 365:725). Up to 50% of patients treated with CART expressing an anti-CD19 CAR develop grade 3 or higher CRS and neurotoxicity (see, e.g., Neelapu, et al (2017) NEJM 377:2531; Maude, et al. (2014) NEJM 371:1507; Gust, et al (2017) Cancer Discov. 7:1404). The development of CRS is associated with in vivo T cell expansion and massive production of T cell effector cytokines (e.g., interleukin 6 (IL6), interferon-γ (IFNγ), monocyte chemoattractant protein 1 (MCP1), and/or granulocyte macrophage colony stimulating factor (GM-CSF)) (see, e.g., Neelapu, et al (2017) NEJM 377:2531; Maude, et al. (2014) NEJM 371:1507; Teachey, et al (2016) Cancer Discov. 6:664; Park et al (2018) Clin Infect Dis 67:533).

Accordingly, there remains a need for therapeutic approaches that improve the efficacy and safety of CART therapies for treatment of hematological disorders.

Combination of anti-CD19 CART therapy with other treatments of hematological malignancies has been explored to improve response rates. One such treatment includes the irreversible small molecule BTK inhibitor ibrutinib, which has been approved for treatment of relapsed refractory or treatment naïve patients with CLL/small lymphocytic lymphoma (SLL). In certain B cell malignancies such as mantle cell lymphoma (MCL) and CLL, aberrant activation of the B cell receptor (BCR) signaling pathway is the dominant mechanism underlying oncogenesis (see, e.g., Herve, et al (2005) J. Clin Invest. 115:1636; Contri, et al (2005) J. Clin Invest 115:369; Ringshausen, et al (2002) Blood 100:3741; Muzio, et al (2008) Blood 112). BTK is a key component of proximal BCR signaling that initiates a phosphorylation cascade through NF-kappaB and MAP kinases that promote B cell survival and activation (e.g., aberrant proliferation and activation in the context of malignant B cells). In CLL, BTK expression is upregulated in CLL cells compared to non-malignant B cells, and targeting BTK with ibrutinib results in direct cytotoxicity, inhibition of proliferation, disruption of cytokine/chemokine signaling, and inhibition of cell migration (see, e.g., Herman, et al (2011) Blood 117:6287; Woyach, et al (2014) Blood 123:1207; Ponader, et al (2012) Blood 119:1182; de Rooij, et al (2012) Blood 119:2590).

In CLL patients treated with ibrutinib, inhibition of BTK and ITK by ibrutinib results in desirable functional outcomes for overcoming cancer-associated immunosuppression, e.g., improved in vivo persistence of activated T cells, decreased ratio of immunosuppressive Treg cells to CD4+ T cells, and diminished immune suppressive properties of CLL cells (see, e.g., Long, et al. (2017) J Clin Invest 127:3052). Without being bound by theory, such functional outcomes may alter the phenotype of immune cell compartments in cancer patients in order to improve effector function and/or proliferative response of CAR-expressing cells administered to the cancer patient. Indeed, combination of ibrutinib with CART19 therapy has been shown to improve response rates to CART therapy in certain B cell malignancies, e.g., by improving expansion, proliferation, and/or persistence of the CART cells following administration (see, e.g., Fraietta, et al (2016) Blood 127:1117; Ruella, et al (2016) Clin Cancer Res 22:2684).

However, treatment with ibrutinib is problematic due to disease progression and acquired resistance. Ibrutinib binds to BTK at the cysteine 481 residue (C481) and cysteine mutations at residue 481 (e.g., BTK^{C481S} or BTK^{C481R}) result in impaired BTK binding that result in drug resistance (see, e.g., Furman, et al (2014) NEJM 370:2352; Woyach et al (2014) NEJM 370:2286; Burger, et al (2016) Nat Commun 7:11589; Landau, et al (2017) Nat. Commun 8:2185; Woyach et al (2017) J Clin Oncol 35:1437). Additionally, treatment with ibrutinib is associated with toxicities due, at least in part, to its potent inhibition of BTK-related tyrosine kinases that include endothelial growth factor (EGFR), interleukin-2-inducible T cell kinase (ITK), tyrosine kinase expressed in hepatocellular carcinoma (TEC), and bone marrow tyrosine kinase on chromosome X (BMX). For example, off-target inhibition of EGFR is associated with rash and diarrhea (see, e.g., Bond, et al (2019) Curr. Hematologic Malignancy Reports 14:197). Other treatment-related toxicities associated with ibrutinib include increased risk for atrial fibrillation (see, e.g., Byrd, et al (2013) NEJM 369:32; Burger et al (2015) NEJM 373:2425; Brown, et al (2017) Haematologica 102:1796; Ganatra, et al (2018) JACC Clin Electrophysiol 4:1491; Leong, et al (2016) Blood 128:138) and increased risk for bleeding (see, e.g., Byrd, et al (2014) NEJM 371:213; Byrd, et al (2013) NEJM 369:1278).

Due to the limitations of ibrutinib, there remains an unmet need for treatments that can be safely combined with CART therapy to improve response rates in patients with hematological malignancies. Vecabrutinib (also referred to as “SNS-062”) is a potent and selective reversible inhibitor of BTK and ITK that has been shown to maintain inhibitory activity against both wild-type and mutant BTK (see, e.g., Aslan, et al (2021) Blood Adv 5:3134). For example, clinical trials with vecabrutinib in B cell malignancies have demonstrated that it has efficacy in CLL patients expressing BTK^{C481S} (see, e.g., Allan, et al (2019) Blood 134(Supplement 1):3041). As demonstrated herein, the combination of vecabrutinib with CART19 cells was shown to potentiate in vitro killing of tumor cell expressing CD19. Unlike ibrutinib which demonstrates dose-dependent toxicity to CART cells, vecabrutinib was not toxic to CART19 cells at concentrations up to 10 μM. Indeed, higher concentrations of vecabrutinib were demonstrated to increase in vitro proliferation of CART19 cells and maintain CART19 functionality (e.g., degranulation) in the presence of CD19-expressing tumor cells. Moreover, it was demonstrated that CART19 cells treated with vecabrutinib in the presence of CD19-expressing tumor cells have dampened expression of cytokines associated with CRS.

Accordingly, the present disclosure provides methods and compositions for improving the efficacy and safety of CART therapies comprising administering the CART therapy in combination with a BTK inhibitor described herein (e.g., vecabrutinib). The BTK inhibitors of the disclosure provide a solution to the shortcomings of CART combination therapy with ibrutinib for treating hematological malignancies. For example, the BTK inhibitors of the disclosure (e.g., vecabrutinib) are potent inhibitors of BTK and ITK. Without being bound by theory, inhibition of both BTK and ITK reduces immune suppression in cancer patients, thereby favoring CART cell effector function and proliferation in vivo. In addition, the BTK inhibitors of the disclosure (e.g., vecabrutinib) are selective inhibitors (e.g., low potency for tyrosine kinase that are not BTK or ITK), thereby reducing the risk of treatment-related toxicities. Additionally, they are potent inhibiters of both wild-type BTK and BTK mutants that result from ibrutinib treatment (e.g., BTK^{C481S} or BTK^{C481R}). Accordingly, and without being bound by theory, the BTK inhibitors of the disclosure are effective in patients who have developed resistance to ibrutinib therapy via BTK mutations at residue 481.

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the preparation of the combinations described herein.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps order for performing certain actions is immaterial so long as the invention remains operable. Moreover, two or more steps or actions can be conducted simultaneously.

It is to be understood that, unless otherwise stated, any description of a method of treatment or prevention includes use of the combination to provide such treatment or prevention as is described herein. It is to be further understood, unless otherwise stated, any description of a method of treatment or prevention includes use of the combination to prepare a medicament to treat or prevent such condition. The treatment or prevention includes treatment or prevention of human or non-human animals including rodents and other disease models.

It is to be understood that, unless otherwise stated, any description of a method of treatment includes use of the combination to provide such treatment as is described herein. It is to be further understood, unless otherwise stated, any description of a method of treatment includes use of the combination to prepare a medicament to treat such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a combination of the present disclosure, or a pharmaceutically acceptable salt, polymorph or solvate of the inhibitor thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a combination of the present disclosure, or a pharmaceutically acceptable salt, polymorph or solvate of the inhibitor thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that the present disclosure also provides pharmaceutical compositions comprising any combination described herein in combination with at least one pharmaceutically acceptable excipient or carrier.

As used herein, the term “pharmaceutical composition” is a formulation containing the combination of the present disclosure in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed peptide or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a peptide of this disclosure include powders, sprays, ointments, pastes, creams, lotions, gels, solutions, patches and inhalants. In some embodiments, the active peptide is mixed under sterile conditions with a pharmaceutically acceptable carrier, and with any preservatives, buffers, or propellants that are required.

As used herein, the term “pharmaceutically acceptable” refers to those combinations, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a combination or pharmaceutical composition of the disclosure can be administered to a subject in many of the well-known methods currently used for chemotherapeutic treatment. For example, a combination of the disclosure may be injected into the blood stream or body cavities. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

As used herein, the term “therapeutically effective amount”, refers to an amount of a pharmaceutical agent to treat or ameliorate an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any combination, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans.

Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active combinations of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active peptides into preparations that can be used pharmaceutically. The appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active combination in the required amount in an appropriate solvent with one or a combination of ingredients enumerated above, as required, followed by filtered sterilization. Generally, dispersions are prepared by incorporating the active combination into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, an active component of the combination can be incorporated with excipients and used in the form of tablets, troches, or capsules. Oral compositions can also be prepared using a fluid carrier for use as a mouthwash, wherein the peptide in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or peptides of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active peptides are formulated into ointments, salves, gels, or creams as generally known in the art.

The active combination can be prepared with pharmaceutically acceptable carriers that will protect against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active peptide to produce the desired biological effect in a subject or cell.

It is to be understood that the pharmaceutical compositions can be included in a container, pack, or dispenser together with instructions for administration.

It is to be understood that, for the inhibitors (e.g., vecabrutinib) of the present disclosure being capable of further forming salts, all of these forms are also contemplated within the scope of the claimed disclosure.

As used herein, the term “pharmaceutically acceptable salts” refer to derivatives of the inhibitors (e.g., vecabrutinib) of the present disclosure wherein the parent inhibitor is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent peptide formed, for example, from non-toxic inorganic organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent peptide either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the peptide to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The combinations, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. One skilled in the art will recognise the advantages of certain routes of administration.

The dosage regimen utilizing the combination is selected in accordance with a variety of factors including type, species, age, weight, sex and medical condition of the patient; the severity of the condition to be treated; the route of administration; the renal and hepatic function of the patient; and the particular peptide or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed peptides of the disclosure can be found in Remington: the Science and Practice of Pharmacy, 19^{th} edition, Mack Publishing Co., Easton, PA (1995). In some embodiments, the combinations described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous organic solutions. The combination will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “combination of the disclosure” refers to those combinations which are disclosed herein, both generically and specifically.

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule.

A therapy that comprises a CAR-expressing cell is referred to herein as a CAR-therapy. For example, a therapy that comprises a CD19 CAR may be referred to herein as a “CD19 CAR therapy” or a “CART19 therapy.”

As used herein, the term “CD19” refers to the Cluster of Differentiation 19 protein, which is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM_001178098. As used herein, “CD19” includes proteins comprising mutations, e.g., point mutations, fragments, insertions, deletions and splice variants of full length wild-type CD19. CD19 is expressed on most B lineage cancers, including, e.g., acute lymphoblastic leukemia, chronic lymphocyte leukemia and non-Hodgkin lymphoma. In some aspects the antigen-binding portion of the CART recognizes and binds an antigen within the extracellular domain of the CD19 protein. In some aspects, the CD19 protein is expressed on a cancer cell.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen binding domain, that is sufficient to confer recognition and specific binding of the antibody fragment to a target, such as an antigen.

The term “combination” refers to either a fixed combination in one dosage unit form, or a combined administration where a compound of the present disclosure and a combination partner may be administered independently at the same time or separately within time intervals.

The terms “co-administration” or “combined administration” as utilized herein are meant to encompass administration of the selected combination partner to a single subject in need thereof, and include treatment regimens in which the agents are not necessarily administered by the same route of administration or at the same time.

The term “nucleic acid,” “polynucleotide,” or “nucleic acid molecule” refers to deoxyribonucleic acid (DNA) or ribonucleic acid (RNA), or a combination of a DNA or RNA thereof, and polymers thereof in either single- or double-stranded form. The term “nucleic acid” includes a gene, cDNA or an mRNA. In some embodiments, the nucleic acid molecule is synthetic (e.g., chemically synthesized) or recombinant. In some embodiments, the term encompasses nucleic acids containing analogues or derivatives of natural nucleotides that have similar binding properties as the reference nucleic acid and are metabolized in a manner similar to naturally occurring nucleotides.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

As used herein, the terms “cancer” (or “cancerous”), “hyperproliferative,” and “neoplastic” refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The combination therapies provided herein can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

Additional examples of proliferative disorders include hematological malignancies.

As used herein, the term “hematological malignancy” refers to diseases involving hyperplastic, neoplastic, and/or malignant cells of hematopoietic origin, e.g., arising from myeloid, lymphoid or erythroid lineages, or precursor cells thereof. In some embodiments, the diseases arise from poorly differentiated acute leukemias (e.g., erythroblastic leukemia and acute megakaryoblastic leukemia). Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macro globulinemia (WM). Additional forms of malignant lymphomas include, but are not limited to, non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin's disease and Reed-Sternberg disease.

### Combination Therapy

The present disclosure provides a combination therapy comprising a BTK inhibitor described herein (e.g., vecabrutinib) and an immunotherapy for use in treating or preventing a disease or disorder (e.g., cancer) in a subject.

The present disclosure provides a combination therapy comprising a BTK inhibitor described herein (e.g., vecabrutinib) and an immunotherapy for use in treating a disease or disorder (e.g., cancer) in a subject.

In some embodiments, the immunotherapy is an adoptive immune cell therapy. In some embodiments, the adoptive immune cell therapy comprises a population of immune cells (e.g., T cells or NK cells) engineered to express a CAR that recognizes and/or targets an antigen associated with the disease or disorder. In some embodiments, the adoptive immune cell therapy comprises T cells engineered to express a CAR that recognizes and/or targets an antigen associated with the disease or disorder.

In some embodiments, the disclosure provides a combination therapy comprising a BTK inhibitor described herein (e.g., vecabrutinib) and an immunotherapy (e.g., CAR therapy) for use in treating or preventing a cancer in a subject.

In some embodiments, the disclosure provides a combination therapy comprising a BTK inhibitor described herein (e.g., vecabrutinib) and an immunotherapy (e.g., CAR therapy) for use in treating a cancer in a subject.

In some embodiments, the cancer comprises a hematological malignancy. In some embodiments, the hematological malignancy comprises a myeloma, a lymphoma, or a leukemia. In some embodiments, the hematological malignancy comprises a B cell malignancy described herein.

In some embodiments, the immunotherapy is an adoptive immune cell therapy comprising a population of immune cells (e.g., T cells or NK cells) engineered to express a CAR that recognizes and/or targets an antigen associated with the cancer (e.g., a hematological malignancy and/or a B cell malignancy). Exemplary antigens are further described herein.

In some embodiments, the immunotherapy is an adoptive immune cell therapy comprising a population of immune cells (e.g., T cells or NK cells) engineered to express a CAR that recognizes and/or targets a B cell antigen associated with a hematological malignancy and/or a B cell malignancy. Exemplary B cell antigens are further described herein. In some embodiments, the B cell antigen is CD19.

In some embodiments, the efficacy of the adoptive immune cell therapy (e.g., CAR therapy, e.g., CART therapy) for treating or preventing a disease or disorder (e.g., cancer) in a subject depends upon one or more in vivo functions of the immune cells (e.g., CAR-expressing immune cells, e.g., CAR-expressing T cells) that are administered to the subject, such as the ability of the immune cells to (i) localize to diseased tissues (e.g., cancerous tissues); (ii) recognize and bind to a target antigen expressed on diseased cells (e.g., tumor cells); (iii) undergo activation, expansion, and exert effector functions upon recognition of a target antigen (e.g., cytotoxic killing and/or secretion of inflammatory cytokines); (iv) differentiate, transition, or reprogram into desirable phenotypic states (e.g., long-lived memory, less differentiated, and/or effector states); (v) avoid exhaustion, anergy, and/or differentiation into a suppressive state, e.g., as a result of the immunosuppressive conditions within the diseased tissue microenvironment (e.g., tumor microenvironment); and/or (vi) undergo a robust memory response following re-exposure to target antigen.

In some embodiments, the efficacy of the adoptive immune cell therapy (e.g., CAR therapy, e.g., CART therapy) for treating a disease or disorder (e.g., cancer) in a subject depends upon one or more in vivo functions of the immune cells (e.g., CAR-expressing immune cells, e.g., CAR-expressing T cells) that are administered to the subject, such as the ability of the immune cells to (i) localize to diseased tissues (e.g., cancerous tissues); (ii) recognize and bind to a target antigen expressed on diseased cells (e.g., tumor cells); (iii) undergo activation, expansion, and exert effector functions upon recognition of a target antigen (e.g., cytotoxic killing and/or secretion of inflammatory cytokines); (iv) differentiate, transition, or reprogram into desirable phenotypic states (e.g., long-lived memory, less differentiated, and/or effector states); (v) avoid exhaustion, anergy, and/or differentiation into a suppressive state, e.g., as a result of the immunosuppressive conditions within the diseased tissue microenvironment (e.g., tumor microenvironment); and/or (vi) undergo a robust memory response following re-exposure to target antigen.

In some embodiments, the BTK inhibitor described herein (e.g., vecabrutinib) is administered in combination with the adoptive immune cell therapy (e.g., CAR therapy, e.g., a CART therapy) to improve one or more in vivo functions of the immune cells (e.g., CAR-expressing immune cells, e.g., CAR-expressing T cells), e.g., as compared to the adoptive immune cell therapy administered without the BTK inhibitor.

In some embodiments, the BTK inhibitor described herein (e.g., vecabrutinib) is administered in combination with the adoptive immune cell therapy (e.g., CAR therapy, e.g., a CART therapy) to provide increased expansion of the immune cells (e.g., CAR-expressing cells, e.g., CAR-expressing T cells) in vivo in the subject, e.g., as compared to the adoptive immune cell therapy administered without the BTK inhibitor. In some embodiments, the increased expansion of the immune cells (e.g., CAR-expressing cells, e.g., CAR-expressing T cells) results without substantially compromising their effector function (e.g., lytic function and/or cytokine expression).

In some embodiments, the BTK inhibitor described herein (e.g., vecabrutinib) is administered in combination with the adoptive immune cell therapy (e.g., CAR therapy, e.g., a CART therapy) to provide increased proliferation of the immune cells (e.g., CAR-expressing cells, e.g., CAR-expressing T cells) in vivo in the subject, e.g., as compared to the adoptive immune cell therapy administered without the BTK inhibitor. In some embodiments, the increased proliferation of the immune cells (e.g., CAR-expressing cells, e.g., CAR-expressing T cells) results without substantially compromising their effector function (e.g., lytic function and/or cytokine expression).

In some embodiments, the BTK inhibitor described herein (e.g., vecabrutinib) is administered in combination with the adoptive immune cell therapy (e.g., CAR therapy, e.g., a CART therapy) to reduce immune suppression of the immune cells (e.g., CAR-expressing cells, e.g., CAR-expressing T cells) in vitro in the subject, e.g., as compared to the adoptive immune cell therapy administered without the BTK inhibitor. In some embodiments, the reduced immune suppression of the immune cells (e.g., CAR-expressing immune cells, e.g., CAR-expressing T cells) results in improved effector function (e.g., lytic function and/or cytokine expression).

In some embodiments, the BTK inhibitor described herein (e.g., vecabrutinib) administered in combination with a CAR therapy described herein (e.g., CART therapy, e.g., CART19 therapy) results in increased expansion of the CAR-expressing cells (e.g., CAR-expressing T cells), increased proliferation of the CAR-expressing cells (e.g., CAR-expressing T cells), and/or reduced immune suppression of the CAR-expressing cells (e.g., CAR-expressing T cells) without reducing the ability of the CART cells to become activated, undergo degranulation, secrete one or more desired cytokines or chemokines, and/or persist.

Methods known in the art and described in the Exemplary section provided herein are suitable for measuring the functionality of CAR-expressing cells in the presence of the BTK inhibitor, and include in vitro assays to compare proliferation, target cell killing, and/or cytokine expression in culture conditions that in the presence and in the absence of the inhibitor.

### BTK Inhibitors

In some embodiments, the BTK inhibitor is a small molecule.

In some embodiments, the BTK inhibitor is a reversible inhibitor of BTK.

In some embodiments, the BTK inhibitor inhibits BTK with a half-maximal inhibitor concentration (IC₅₀) of less than about 100 nM, about 90 nM, about 80 nM, about 70 nM, about 60 nM, about 50 nM, about 40 nM, about 30 nM, about 20 nM, about 15 nM, about 10 nM, about 9 nM, about 8 nM, about 7 nM, about 6 nM, about 5 nM, about 4 nM, about 3 nM, about 2 nM, or about 1 nM.

In some embodiments, the BTK inhibitor inhibits BTK with a half-maximal inhibitor concentration (IC₅₀) of less than about 100 nM.

In some embodiments, the BTK inhibitor inhibits BTK with a half-maximal inhibitor concentration (IC₅₀) of less than about 90 nM.

In some embodiments, the BTK inhibitor inhibits BTK with a half-maximal inhibitor concentration (IC₅₀) of less than about 80 nM.

In some embodiments, the BTK inhibitor inhibits BTK with a half-maximal inhibitor concentration (IC₅₀) of less than about 70 nM.

In some embodiments, the BTK inhibitor inhibits BTK with a half-maximal inhibitor concentration (IC₅₀) of less than about 60 nM.

In some embodiments, the BTK inhibitor inhibits BTK with a half-maximal inhibitor concentration (IC₅₀) of less than about 50 nM.

In some embodiments, the BTK inhibitor inhibits BTK with a half-maximal inhibitor concentration (IC₅₀) of less than about 40 nM.

In some embodiments, the BTK inhibitor inhibits BTK with a half-maximal inhibitor concentration (IC₅₀) of less than about 30 nM.

In some embodiments, the BTK inhibitor inhibits BTK with a half-maximal inhibitor concentration (IC₅₀) of less than about 20 nM.

In some embodiments, the BTK inhibitor inhibits BTK with a half-maximal inhibitor concentration (IC₅₀) of less than about 15 nM.

In some embodiments, the BTK inhibitor inhibits BTK with a half-maximal inhibitor concentration (IC₅₀) of less than about 10 nM.

In some embodiments, the BTK inhibitor inhibits BTK with a half-maximal inhibitor concentration (IC₅₀) of less than about 9 nM.

In some embodiments, the BTK inhibitor inhibits BTK with a half-maximal inhibitor concentration (IC₅₀) of less than about 8 nM.

In some embodiments, the BTK inhibitor inhibits BTK with a half-maximal inhibitor concentration (IC₅₀) of less than about 7 nM.

In some embodiments, the BTK inhibitor inhibits BTK with a half-maximal inhibitor concentration (IC₅₀) of less than about 6 nM.

In some embodiments, the BTK inhibitor inhibits BTK with a half-maximal inhibitor concentration (IC₅₀) of less than about 5 nM.

In some embodiments, the BTK inhibitor inhibits BTK with a half-maximal inhibitor concentration (IC₅₀) of less than about 4 nM.

In some embodiments, the BTK inhibitor inhibits BTK with a half-maximal inhibitor concentration (IC₅₀) of less than about 3 nM.

In some embodiments, the BTK inhibitor inhibits BTK with a half-maximal inhibitor concentration (IC₅₀) of less than about 2 nM.

In some embodiments, the BTK inhibitor inhibits BTK with a half-maximal inhibitor concentration (IC₅₀) of less than about 1 nM.

In some embodiments, the BTK inhibitor is a reversible inhibitor of a mutant BTK, wherein the mutant BTK comprises a mutation at residue 481, and wherein the mutation is selected from C481R and C481S.

In some embodiments, the BTK inhibitor inhibits wild-type BTK and mutant BTK (e.g., BTK^{C481R} or BTK^{C481S}), wherein the BTK inhibitor has a half-maximal inhibitor concentration (IC₅₀) for wild-type BTK that is substantially equivalent (e.g., ±1%, ±2%, ±3%, ±4%, ±5%, ±6%, ±7%, ±8%, ±9%, ±10%) to its IC₅₀ for mutant BTK.

In some embodiments, the BTK inhibitor inhibits mutant BTK (e.g., BTKC481R or BTKC481S) with a half-maximal inhibitor concentration (IC₅₀) of less than about 100 nM, about 90 nM, about 80 nM, about 70 nM, about 60 nM, about 50 nM, about 40 nM, about 30 nM, about 20 nM, about 15 nM, about 10 nM, about 9 nM, about 8 nM, about 7 nM, about 6 nM, about 5 nM, about 4 nM, about 3 nM, about 2 nM, or about 1 nM.

In some embodiments, the BTK inhibitor inhibits mutant BTK (e.g., BTKC481R or BTKC481S) with a half-maximal inhibitor concentration (IC₅₀) of less than about 100 nM.

In some embodiments, the BTK inhibitor inhibits mutant BTK (e.g., BTKC481R or BTKC481S) with a half-maximal inhibitor concentration (IC₅₀) of less than about 90 nM.

In some embodiments, the BTK inhibitor inhibits mutant BTK (e.g., BTKC481R or BTKC481S) with a half-maximal inhibitor concentration (IC₅₀) of less than about 80 nM.

In some embodiments, the BTK inhibitor inhibits mutant BTK (e.g., BTKC481R or BTKC481S) with a half-maximal inhibitor concentration (IC₅₀) of less than about 70 nM.

In some embodiments, the BTK inhibitor inhibits mutant BTK (e.g., BTKC481R or BTKC481S) with a half-maximal inhibitor concentration (IC₅₀) of less than about 60 nM.

In some embodiments, the BTK inhibitor inhibits mutant BTK (e.g., BTKC481R or BTKC481 S) with a half-maximal inhibitor concentration (IC₅₀) of less than about 50 nM.

In some embodiments, the BTK inhibitor inhibits mutant BTK (e.g., BTKC481R or BTKC481S) with a half-maximal inhibitor concentration (IC₅₀) of less than about 40 nM.

In some embodiments, the BTK inhibitor inhibits mutant BTK (e.g., BTKC481R or BTKC481S) with a half-maximal inhibitor concentration (IC₅₀) of less than about 30 nM.

In some embodiments, the BTK inhibitor inhibits mutant BTK (e.g., BTKC481R or BTKC481S) with a half-maximal inhibitor concentration (IC₅₀) of less than about 20 nM.

In some embodiments, the BTK inhibitor inhibits mutant BTK (e.g., BTKC481R or BTKC481S) with a half-maximal inhibitor concentration (IC₅₀) of less than about 15 nM.

In some embodiments, the BTK inhibitor inhibits mutant BTK (e.g., BTKC481R or BTKC481S) with a half-maximal inhibitor concentration (IC₅₀) of less than about 10 nM.

In some embodiments, the BTK inhibitor inhibits mutant BTK (e.g., BTKC481R or BTKC481S) with a half-maximal inhibitor concentration (IC₅₀) of less than about 9 nM.

In some embodiments, the BTK inhibitor inhibits mutant BTK (e.g., BTKC481R or BTKC481S) with a half-maximal inhibitor concentration (IC₅₀) of less than about 8 nM.

In some embodiments, the BTK inhibitor inhibits mutant BTK (e.g., BTKC481R or BTKC481S) with a half-maximal inhibitor concentration (IC₅₀) of less than about 7 nM.

In some embodiments, the BTK inhibitor inhibits mutant BTK (e.g., BTKC481R or BTKC481S) with a half-maximal inhibitor concentration (IC₅₀) of less than about 6 nM.

In some embodiments, the BTK inhibitor inhibits mutant BTK (e.g., BTKC481R or BTKC481S) with a half-maximal inhibitor concentration (IC₅₀) of less than about 5 nM.

In some embodiments, the BTK inhibitor inhibits mutant BTK (e.g., BTKC481R or BTKC481S) with a half-maximal inhibitor concentration (IC₅₀) of less than about 4 nM.

In some embodiments, the BTK inhibitor inhibits mutant BTK (e.g., BTKC481R or BTKC481S) with a half-maximal inhibitor concentration (IC₅₀) of less than about 3 nM.

In some embodiments, the BTK inhibitor inhibits mutant BTK (e.g., BTKC481R or BTKC481S) with a half-maximal inhibitor concentration (IC₅₀) of less than about 2 nM.

In some embodiments, the BTK inhibitor inhibits mutant BTK (e.g., BTKC481R or BTKC481S) with a half-maximal inhibitor concentration (IC₅₀) of less than about 1 nM.

In some embodiments, the BTK inhibitor is a reversible inhibitor of BTK and ITK.

In some embodiments, the BTK inhibitor inhibits ITK with a half-maximal inhibitor concentration (IC₅₀) of less than about 100 nM, about 90 nM, about 80 nM, about 70 nM, about 60 nM, about 50 nM, about 40 nM, about 30 nM, about 20 nM, about 15 nM, about 10 nM, about 9 nM, about 8 nM, about 7 nM, about 6 nM, about 5 nM, about 4 nM, about 3 nM, about 2 nM, or about 1 nM.

In some embodiments, the BTK inhibitor inhibits ITK with a half-maximal inhibitor concentration (IC₅₀) of less than about 100 nM.

In some embodiments, the BTK inhibitor inhibits ITK with a half-maximal inhibitor concentration (IC₅₀) of less than about 90 nM.

In some embodiments, the BTK inhibitor inhibits ITK with a half-maximal inhibitor concentration (IC₅₀) of less than about 80 nM.

In some embodiments, the BTK inhibitor inhibits ITK with a half-maximal inhibitor concentration (IC₅₀) of less than about 70 nM.

In some embodiments, the BTK inhibitor inhibits ITK with a half-maximal inhibitor concentration (IC₅₀) of less than about 60 nM.

In some embodiments, the BTK inhibitor inhibits ITK with a half-maximal inhibitor concentration (IC₅₀) of less than about 50 nM.

In some embodiments, the BTK inhibitor inhibits ITK with a half-maximal inhibitor concentration (IC₅₀) of less than about 40 nM.

In some embodiments, the BTK inhibitor inhibits ITK with a half-maximal inhibitor concentration (IC₅₀) of less than about 30 nM.

In some embodiments, the BTK inhibitor inhibits ITK with a half-maximal inhibitor concentration (IC₅₀) of less than about 20 nM.

In some embodiments, the BTK inhibitor inhibits ITK with a half-maximal inhibitor concentration (IC₅₀) of less than about 15 nM.

In some embodiments, the BTK inhibitor inhibits ITK with a half-maximal inhibitor concentration (IC₅₀) of less than about 10 nM.

In some embodiments, the BTK inhibitor inhibits ITK with a half-maximal inhibitor concentration (IC₅₀) of less than about 9 nM.

In some embodiments, the BTK inhibitor inhibits ITK with a half-maximal inhibitor concentration (IC₅₀) of less than about 8 nM.

In some embodiments, the BTK inhibitor inhibits ITK with a half-maximal inhibitor concentration (IC₅₀) of less than about 7 nM.

In some embodiments, the BTK inhibitor inhibits ITK with a half-maximal inhibitor concentration (IC₅₀) of less than about 6 nM.

In some embodiments, the BTK inhibitor inhibits ITK with a half-maximal inhibitor concentration (IC₅₀) of less than about 5 nM.

In some embodiments, the BTK inhibitor inhibits ITK with a half-maximal inhibitor concentration (IC₅₀) of less than about 4 nM.

In some embodiments, the BTK inhibitor inhibits ITK with a half-maximal inhibitor concentration (IC₅₀) of less than about 3 nM.

In some embodiments, the BTK inhibitor inhibits ITK with a half-maximal inhibitor concentration (IC₅₀) of less than about 2 nM.

In some embodiments, the BTK inhibitor inhibits ITK with a half-maximal inhibitor concentration (IC₅₀) of less than about 1 nM.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for one or more TEC family kinase inhibitors selected from TXK, BMK, EGFR, ERBB2, ERBB4, CSK, FGR, BRK, HCK, YES, JAK3, FRK, RET, FLT3, ABL and FYN.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for TXK.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for BMK.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for EGFR.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for ERBB2.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for ERBB4.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for CSK.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for FGR.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for BRK.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for HCK.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for YES.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for JAK3.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for FRK.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for RET.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for FLT3.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for ABL.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for FYN.

In some embodiments, the IC₅₀ is measured or determined using an in vitro assay. Assays to assess or quantitate or measure activity of protein tyrosine kinase inhibitors as described are known in the art. Such assays can be conducted in vitro and include assays to assess the ability of an agent to inhibit a specific biological or biochemical function. In some embodiments, kinase activity studies can be performed. Protein tyrosine kinases catalyze the transfer of the terminal phosphate group from adenosine triphosphate (ATP) to the hydroxyl group of a tyrosine residue of the kinase itself or another protein substrate. In some embodiments, kinase activity can be measured by incubating the kinase with the substrate (e.g., inhibitor) in the presence of ATP. In some embodiments, measurement of the phosphorylated substrate by a specific kinase can be assessed by several reporter systems including colorimetric, radioactive, and fluorometric detection. (Johnson, S. A. & T. Hunter (2005) Nat. Methods 2:17.) In some embodiments, inhibitors can be assessed for their affinity for a particular kinase or kinases, such as by using competition ligand binding assays (Ma et al., Expert Opin Drug Discov. 2008 June; 3(6):607-621) From these assays, the half-maximal inhibitory concentration (IC₅₀) can be calculated. IC₅₀ is the concentration that reduces a biological or biochemical response or function by 50% of its maximum. In some cases, such as in kinase activity studies, IC₅₀ is the concentration of the compound that is required to inhibit the target kinase activity by 50%. In some cases, the dissociation constant (K_{d}) and/or the inhibition constant (Kᵢ values) can be determined additionally or alternatively. IC₅₀ and K_{d} can be calculated by any number of means known in the art. The inhibition constant (Kᵢ values) can be calculated from the IC₅₀ and Kd values according to the Cheng-Prusoff equation: Kᵢ=IC₅₀/(l+L/K_{d}), where L is the concentration of the inhibitor (Biochem Pharmacol 22: 3099-3108, 1973). Kᵢ is the concentration of unlabeled inhibitor that would cause occupancy of 50% of the binding sites present in the absence of ligand or other competitors.

PCT patent publication WO2013/185084 (PCT application PCT/US13/44800, filed Jun. 7, 2013), the entirety of which is hereby incorporated herein by reference, describes certain BTK inhibitor compounds. Such compounds include (3R,3′R,4′S)-1′-(6-amino-5-fluoropyrimidin-4-yl)-3-((3-chloro-5-(trifluoromethyl) phenyl)amino)-2-oxo-[1,3′-bipiperidine]-4′-carboxamide:

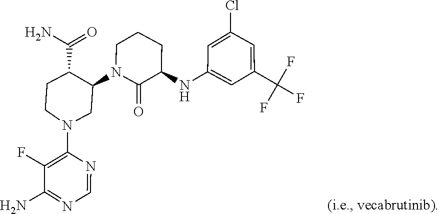 [SMILES: NC(=O)[C@H]1CCN(c2ncnc(N)c2F)C[C@@H]1N1CCC[C@@H](Nc2cc(Cl)cc(C(F)(F)F)c2)C1=O]

In some embodiments, the BTK inhibitor is vecabrutinib, or a pharmaceutically acceptable salt thereof.

In some embodiments, vecabrutinib is provided in a solid form that imparts characteristics such as improved aqueous solubility, stability, absorption, bioavailability, and ease of formulation and isolation.

In some embodiments, the pharmaceutically acceptable salt of vecabrutinib is the succinic acid.

In some embodiments, the succinic acid salt of vecabrutinib depicted as:

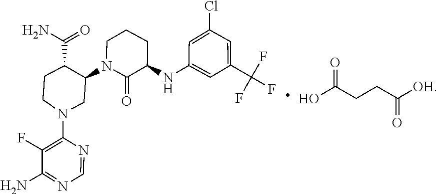 [SMILES: NC(=O)[C@H]1CCN(c2ncnc(N)c2F)C[C@@H]1N1CCC[C@@H](Nc2cc(Cl)cc(C(F)(F)F)c2)C1=O ; O=C(O)CCC(=O)O]

In some embodiments, the succinic acid salt of vecabrutinib has a stoichiometry of (vecabrutinib):(succinic acid) that is about 1:1.

### CAR Molecules

In some aspects, the disclosure provides compositions and methods to be used or performed in combination with CAR-expressing cells.

CAR molecules are genetically-engineered, artificial transmembrane receptors, which confer an arbitrary specificity for a ligand onto an immune effector cell (e.g. a T cell, NK cell, macrophage, or other immune cell) and which results in activation of the effector cell upon recognition and binding to the ligand. For example, in some embodiments, a T cell is engineered to express a CAR molecule on its cell surface that imparts the antigen specificity of a monoclonal antibody and renders the engineered T cell capable of exerting effector functions upon recognition of a target cell with an antigen recognized by the CAR molecule.

In some embodiments, the CAR molecule comprises (i) an ectodomain comprising an antigen binding domain (e.g., CD19 binding domain), (ii) a transmembrane domain, and (iii) an endodomain comprising one or more intracellular signaling domains (e.g., an intracellular signaling domain comprising a costimulatory domain and/or a primary signaling domain). In some embodiments, the ectodomain of the CAR molecule resides outside of the CAR-expressing cell and is exposed to the extracellular space, whereby it is accessible for interaction with its cognate target antigen. The transmembrane domain allows the CAR molecule to be anchored into the cell membrane of the cell engineered to express the CAR molecule. The endodomain (also known as the “activation domain”) aids in activation of the CAR-expressing cell upon binding of the CAR to its target antigen. In some embodiments, activation of the CAR-expressing cell comprises induction of cytokine and chemokine production, as well as activation of its cytolytic activity. In some embodiments, the CAR molecule functions to redirect cytotoxicity toward tumor cells expressing the target antigen.

In some embodiments, CARs comprise an antigen binding domain that binds to a specific target antigen. In some embodiments, the antigen binding domain comprises a single-chain antibody variable fragment (scFv), a tethered ligand or the extracellular domain of a co-receptor, fused to a transmembrane domain, which is linked, in turn, to a signaling domain. In some embodiments, the signaling domain is derived from CD3ζ or FcRγ. In some embodiments, the CAR molecule comprises one or more co-stimulatory domains derived from a protein such as CD28, CD137 (also known as 4-1BB), CD134 (also known as OX40) and CD278 (also known as ICOS).

Engagement of the antigen binding domain of the CAR molecule with its target antigen on the surface of a target cell results in clustering of the CAR molecule and delivers an activation stimulus to the CAR-expressing cell. In some embodiments, the main characteristic of CAR molecules are their ability to redirect specificity of the CAR-expressing cell, thereby triggering proliferation, cytokine production, phagocytosis or production of molecules that can mediate cell death of the target antigen by the CAR-expressing cell in a major histocompatibility (MHC) independent manner by exploiting the cell specific targeting abilities of monoclonal antibodies, soluble ligands or cell specific co-receptors. Although scFv-based CARs engineered to contain a signaling domain from CD3ζ or FcRγ have been shown to deliver a potent signal for T cell activation and effector function, they are not sufficient to elicit signals that promote T cell survival and expansion in the absence of a concomitant co-stimulatory signal. A new generation of CARs containing a binding domain, a hinge, a transmembrane and the signaling domain derived from CD3ζ or FcRγ together with one or more co-stimulatory signaling domains (e.g., intracellular co-stimulatory domains derived from CD28, CD137, CD134 and CD278) has been shown to more effectively direct antitumor activity as well as increased cytokine secretion, lytic activity, survival and proliferation in CAR expressing T cells in vitro, in animal models and cancer patients (Milone et al., Molecular Therapy, 2009; 17:1453-1464; Zhong et al., Molecular Therapy, 2010; 18: 413-420; Carpenito et al., PNAS, 2009; 106:3360-3365).

In some embodiments, a CAR molecule of the disclosure comprises an antigen-binding domain, a transmembrane domain, and a cytoplasmic signaling domain. In some embodiments, the cytoplasmic signaling domain comprises a cytoplasmic sequence of CD3ζ sequence sufficient to stimulate a T cell when the antigen-binding domain binds to the antigen, and optionally, a cytoplasmic sequence of one or more (e.g., two, three, or four) co-stimulatory proteins (e.g., a cytoplasmic sequence of one or more of B7-H3, BTLA, CD2, CD7, CD27, CD28, CD30, CD40, CD40L, CD80, CD160, CD244, ICOS, LAG3, LFA-1, LIGHT, NKG2C, 4-1BB, OX40, PD-1, PD-L1, TIM3, and a ligand that specifically binds to CD83) that provides for co-stimulation of the T cell when the antigen-binding domain binds to the antigen. In some embodiments, a CAR can further include a linker. Non-limiting aspects and features of CARs are described below. Additional aspects of CARs and CAR cells, including exemplary antigen binding domains, linkers, transmembrane domains, and cytoplasmic signaling domains, are described in, e.g., Kakarla et al., Cancer J. 20:151-155, 2014; Srivastava et al., Trends Immunol. 36:494-502, 2015; Nishio et al., Oncoimmunology 4(2): e988098, 2015; Ghorashian et al., Br. J. Haematol. 169:463-478, 2015; Levine, Cancer Gene Ther. 22:79-84, 2015; Jensen et al., Curr. Opin. Immunol. 33:9-15, 2015; Singh et al., Cancer Gene Ther. 22:95-100, 2015; Li et al., Zhongguo Shi Yan Xue Ye Xue Za Zhi 22:1753-1756, 2014; Gill et al., Immunol. Rev. 263:68-89, 2015; Magee et al., Discov. Med. 18:265-271, 2014; Gargett et al., Front. Pharmacol. 5:235, 2014; Yuan et al., Zhongguo Shi Yan Xue Ye Xue Za Zhi 22:1137-1141, 2014; Pedgram et al., Cancer J. 20:127-133, 2014; Eshhar et al., Cancer J. 20:123-126, 2014; Ramos et al., Cancer J. 20:112-118, 2014; Maus et al., Blood 123:2625-2635, 2014; Jena et al., Curr. Hematol. Malig. Rep. 9:50-56, 2014; Maher et al., Curr. Gene Ther. 14:35-43, 2014; Riches et al., Discov. Med. 16:295-302, 2013; Cheadle et al., Immunol. Rev. 257:83-90, 2014; Davila et al., Int. J. Hematol. 99:361-371, 2014; Xu et al., Cancer Lett. 343:172-178, 2014; Kochenderfer et al., Nat. Rev. Clin. Oncol. 10:267-276, 2013; Hosing et al., Curr. Hematol. Malig. Rep. 8:60-70, 2013; Hombach et al., Curr. Mol. Med. 13:1079-1088, 2013; Xu et al., Leuk. Lymphoma 54:255-260, 2013; Gilham et al., Trends Mol. Med. 18:377-384, 2012; Lipowska-Bhalla et al., Cancer Immunol. Immunother. 61:953-962, 2012; Chmielewski et al., Cancer Immunol. Immunother. 61:1269-1277, 2013; Jena et al., Blood 116:1035-1044, 2010; Dotti et al, Immunology Reviews 257(1): 107-126, 2013; Dai et al., Journal of the National Cancer Institute 108(7): djv439, 2016; Wang and Riviere, Molecular Therapy-Oncolytics 3: 16015, 2016; U.S. Patent Application Publication Nos. 2018/0057609; 2018/0037625; 2017/0362295; 2017/0137783; 2016/0152723, 2016/0206656, 2016/0199412, 2016/0208018, 2015/0232880, 2015/0225480; 2015/0224143; 2015/0224142; 2015/0190428; 2015/0196599; 2015/0152181; 2015/0140023; 2015/0118202; 2015/0110760; 2015/0099299; 2015/0093822; 2015/0093401; 2015/0051266, 2015/0050729; 2015/0024482; 2015/0023937; 2015/0017141; 2015/0017136; 2015/0017120; 2014/0370045; 2014/0370017; 2014/0369977; 2014/0349402; 2014/0328812; 2014/0322275; 2014/0322216; 2014/0322212; 2014/0322183; 2014/0314795; 2014/0308259; 2014/0301993; 2014/0296492; 2014/0294784; 2014/0286973; 2014/0274909; 2014/0274801; 2014/0271635; 2014/0271582; 2014/0271581; 2014/0271579; 2014/0255363; 2014/0242701; 2014/0242049; 2014/0227272; 2014/0219975; 2014/0170114; 2014/0134720; 2014/0134142; 2014/0120622; 2014/0120136; 2014/0106449; 2014/0106449; 2014/0099340; 2014/0086828; 2014/0065629; 2014/0050708; 2014/0024809; 2013/0344039; 2013/0323214; 2013/0315884; 2013/0309258; 2013/0288368; 2013/0287752; 2013/0287748, 2013/0280221; 2013/0280220; 2013/0266551; 2013/0216528; 2013/0202622; 2013/0071414; 2012/0321667; 2012/0302466; 2012/0301448; 2012/0301447; 2012/0060230; 2011/0213288; 2011/0158957; 2011/0104128; 2011/0038836; 2007/0036773; and 2004/0043401. Additional aspects of CARs and CAR cells, including exemplary antigen binding domains, linkers, transmembrane domains, and cytoplasmic signaling domains, are described in WO 2016/168595; WO 12/079000; 2015/0141347; 2015/0031624; 2015/0030597; 2014/0378389; 2014/0219978; 2014/0206620; 2014/0037628; 2013/0274203; 2013/0225668; 2013/0116167; 2012/0230962; 2012/0213783; 2012/0093842; 2012/0071420; 2012/0015888; 2011/0268754; 2010/0297093; 2010/0158881; 2010/0034834; 2010/0015113; 2009/0304657; 2004/0043401; 2014/0322253; 2015/0118208; 2015/0038684; 2014/0024601; 2012/0148552; 2011/0223129; 2009/0257994; 2008/0160607; 2008/0003683; 2013/0121960; 2011/0052554; and 2010/0178276.

### Antigen Binding Domain

In some embodiments, the CAR molecule comprises an antigen-specific recognition domain that is capable of binding an antigen described herein.

Antigen binding domains specifically bind to an antigen (e.g., a tumor associated antigen (TAA), a tumor-specific antigen, or an antigen that is not substantially expressed on a healthy and/or non-cancerous cell or population thereof). In some embodiments, the antigen binding domain (e.g., CD19 binding domain) is characterized by particular functional features or properties of an antibody or antibody fragment. Non-limiting examples of an antigen binding domain include: a monoclonal antibody (e.g., IgG1, IgG2, IgG3, IgG4, IgM, IgE, and IgD) (e.g., a fully human or a chimeric (e.g., a humanized) antibody), an antigen binding fragment of an antibody (e.g., Fab, Fab′, or F(ab′)2 fragments) (e.g., a fragment of a fully human or a chimeric (e.g., humanized) antibody), a diabody, a triabody, a tetrabody, a minibody, a scFv, scFv-Fc, (scFv)2, scFab, bis-scFv, hc-IgG, a BiTE, a single domain antibody (e.g., a VNAR domain or a VhH domain), IgNAR, and a multispecific (e.g., bispecific antibody) antibody.

In some embodiments, an antigen binding domain comprises at least one (e.g., one, two, three, four, five, or six) complementarity determining regions (CDRs) of an antibody (e.g., any of the three CDRs from an immunoglobulin light chain variable domain or any of the three CDRs from an immunoglobulin heavy chain variable domain) that is capable of specifically binding to the target antigen, such as immunoglobulin molecules (e.g., light or heavy chain immunoglobulin molecules) and immunologically-active (antigen-binding) fragments of immunoglobulin molecules.

In some embodiments, an antigen binding domain is a single-chain antibody (e.g., a VNAR domain or a VHH domain, or any of the single-chain antibodies as described herein). In some embodiments, an antigen binding domain is a whole antibody molecule (e.g., a human, humanized, or chimeric antibody) or a multimeric antibody (e.g., a bi-specific antibody).

In some embodiments, antigen-binding domains include antibody fragments and multispecific (e.g., bi-specific) antibodies or antibody fragments. Examples of antibodies and antigen binding fragments thereof include, but are not limited to, single-chain Fvs (scFvs), Fab fragments, Fab′ fragments, F(ab′) 2, disulfide-linked Fvs (sdFvs), Fvs, and fragments containing either a VL or a VH domain. Additional antigen binding domains provided herein are polyclonal, monoclonal, multispecific (multimeric, e.g., bi-specific), human antibodies, chimeric antibodies (e.g., human mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgAi, and IgA2), or subclass. In some embodiments, the antigen binding domain is an IgGi antibody or antigen-binding fragment thereof. In some examples, the antigen binding domain is an IgG4 antibody or antigen-binding fragment thereof. In some embodiments, the antigen binding domain is an immunoglobulin comprising a heavy and light chain.

Additional examples of antigen binding domains are antigen-binding fragments of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4), an antigen-binding fragment of an IgA (e.g., an antigen-binding fragment of IgA1 or IgA2) (e.g., an antigen-binding fragment of a human or humanized IgA, e.g., a human or humanized IgA1 or IgA2), an antigen-binding fragment of an IgD (e.g., an antigen-binding fragment of a human or humanized IgD), an antigen-binding fragment of an IgE (e.g., an antigen-binding fragment of a human or humanized IgE), or an antigen-binding fragment of an IgM (e.g., an antigen-binding fragment of a human or humanized IgM). In some embodiments, an antigen binding domain can bind to a particular antigen (e.g., a tumor-associated antigen) with an affinity (KD) about or less than 1×10⁻⁷ M (e.g., about or less than 1×10⁻⁸ M, about or less than 5×10⁻⁹ M, about or less than 1×10⁻⁹ M, or about or less than 5×10⁻¹⁰ M), e.g., in saline or in phosphate buffered saline.

Methods of making these antigen-binding domains are known in the art. As can be appreciated by those in the art, the choice of the antigen binding domain to include in the CAR depends upon the type and number of target antigens present on the surface of the target cell (e.g., cancer cell). For example, in some embodiments, the antigen binding domain is selected to recognize a target antigen that is a cell surface marker on target cells associated with a particular disease state (e.g., cancer). In some embodiments, cell surface markers that may act as ligands for the antigen binding domain in a CAR of the present disclosure include those associated cancer cells, e.g., cancerous lymphocytes, e.g., cancerous B cells. In some embodiments, the antigen binding domain is chosen to recognize a target antigen that acts as a cell surface marker on cancer cells (e.g., a cell surface marker on cancerous B cells), and/or is a tumor-associated antigen (e.g., a tumor-associated B cell antigen) or a tumor-specific antigen (TSA).

In some embodiments, CAR-expressing cells (e.g., CART cells) comprise a CAR molecule that binds to a tumor antigen (e.g., comprises a tumor antigen binding domain). In some embodiments, the portion of the CAR comprising the antigen binding domain comprises an antigen binding domain that targets a tumor antigen, e.g., a tumor antigen described herein.

In some embodiments, the CAR molecule comprises an antigen binding domain that recognizes a tumor antigen of a hematologic malignancy (e.g., leukemia, acute lymphocytic leukemia, acute myelocytic leukemia, acute promyelocytic leukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera, Hodgkin's disease, non-Hodgkin's disease, multiple myeloma, etc.). In some embodiments, the CAR molecule comprises an antigen binding domain that recognizes a tumor antigen of a B cell malignancy (e.g., non-Hodgkin lymphoma (NHL), an acute lymphoblastic leukemia (ALL), a chronic lymphocytic leukemia (CLL), small lymphocytic lymphoma (SLL), a diffuse large B-cell lymphoma (DLBCL), lymphoplasmacytoid lymphoma (LL), Waldenstrom macroglobulinemia (MG), mantle-cell lymphoma (MCL), follicular lymphoma (FL), marginal zone lymphoma (MZL), acute myeloid leukemia (AML), and a myeloma, e.g., a multiple myeloma (MM)). In some embodiments, the CAR molecule comprises an antigen binding domain that recognizes a tumor antigen of a solid tumor (e.g., breast cancer, colon cancer, etc.).

In some embodiments, the tumor antigen is a tumor-specific antigen (TSA). A TSA is unique to tumor cells and does not occur on other cells in the body. In some embodiments, the tumor antigen is a tumor-associated antigen (TAA). A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. In some embodiments, a TAA is expressed on normal cells during fetal development when the immune system is immature and unable to respond or is normally present at extremely low levels on normal cells but which are expressed at much higher levels on tumor cells.

In some embodiments, the tumor-associated antigen is determined by sequencing a patient's tumor cells and identifying mutated proteins only found in the tumor. These antigens are referred to as “neoantigens.” Once a neoantigen has been identified, therapeutic antibodies can be produced against it and used in the methods described herein.

In some embodiments, the tumor antigen is an epithelial cancer antigen, (e.g., breast, gastrointestinal, lung), a prostate specific cancer antigen (PSA) or prostate specific membrane antigen (PSMA), a bladder cancer antigen, a lung (e.g., small cell lung) cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, or a colorectal cancer antigen. In some embodiments, the tumor antigen is a lymphoma antigen (e.g., non-Hodgkin's lymphoma or Hodgkin's lymphoma), a B-cell lymphoma cancer antigen, a leukemia antigen, a myeloma (e.g., multiple myeloma or plasma cell myeloma) antigen, an acute lymphoblastic leukemia antigen, a chronic myeloid leukemia antigen, or an acute myelogenous leukemia antigen.

Tumor antigens, (e.g. tumor-associated antigens (TAAs) and tumor-specific antigens (TSAs)) that may be targeted by CAR effector cells (e.g., CAR T cells), include, but are not limited to, 1GH-IGK, 43-9F, 5T4, 791Tgp72, acyclophilin C-associated protein, alphafetoprotein (AFP), a-actinin-4, A3, antigen specific for A33 antibody, ART-4, B7, Ba 733, BAGE, BCR-ABL, beta-catenin, beta-HCG, BrE3-antigen, BCA225, BTAA, CA125, CA 15-3\CA 27.29YBCAA, CA195, CA242, CA-50, CAM43, CAMEL, CAP-1, carbonic anhydrase IX, c-Met, CA19-9, CA72-4, CAM 17.1, CASP-8/m, CCCL19, CCCL21, CD1, CD1a, CD2, CD3, CD4, CD5, CD8, CD11 A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD68, CD70, CD70L, CD74, CD79a, CD79b, CD80, CD83, CD95, CD126, CD132, CD133, CD138, CD147, CD154, CDC27, CDK4, CDK4m, CDKN2A, CO-029, CTLA4, CXCR4, CXCR7, CXCL12, HIF-1a, colon-specific antigen-p (CSAp), CEA (CEACAM5), CEACAM6, c-Met, DAM, E2A-PRL, EGFR, EGFRvIII, EGP-1 (TROP-2), EGP-2, ELF2-M, Ep-CAM, fibroblast growth factor (FGF), FGF-5, Flt-1, Flt-3, folate receptor, G250 antigen, Ga733VEpCAM, GAGE, gplOO, GRO-b, H4-RET, HLA-DR, HM1.24, human chorionic gonadotropin (HCG) and its subunits, HER2/neu, HMGB-1, hypoxia inducible factor (HIF-1), HSP70-2M, HST-2, HTgp-175, 1a, IGF-1R, IFN-g, IFN-a, IFN-b, IFN-1, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KSA, KS-1-antigen, KS1-4, LAGE-1a, Le-Y, LDR/FUT, M344, MA-50, macrophage migration inhibitory factor (MIF), MAGE, MAGE-1, MAGE-3, MAGE-4, MAGE-5, MAGE-6, MART-1, MART-2, TRAG-3, mCRP, MCP-1, MIP-1A, MIP-1B, MIF, MG7-Ag, MOV18, MUC1, MUC2, MUC3, MUC4, MUC5ac, MUC13, MUC16, MUM-1/2, MUM-3, MYL-RAR, NB/70K, Nm23H1, NuMA, NCA66, NCA95, NCA90, NY-ESO-1, p15, p16, p185erbB2, p180erbB3, PAM4 antigen, pancreatic cancer mucin, PD1 receptor (PD-1), PD-1 receptor ligand 1 (PD-L1), PD-1 receptor ligand 2 (PD-L2), PI5, placental growth factor, p53, PLAGL2, Pmel17 prostatic acid phosphatase, PSA, PRAME, PSMA, PIGF, ILGF, ILGF-IR, IL-6, IL-25, RCAS1, RS5, RAGE, RANTES, Ras, T101, SAGE, S100, survivin, survivin-2B, SDDCAG16, TA-90\Mac2 binding protein, TAAL6, TAC, TAG-72, TLP, tenascin, TRAIL receptors, TRP-1, TRP-2, TSP-180, TNF-a, Tn antigen, Thomson-Friedenreich antigens, tumor necrosis antigens, tyrosinase, VEGFR, ED-B fibronectin, WT-1, 17-IA-antigen, complement factors C3, C3a, C3b, C5a, C5, an angiogenesis marker, bcl-2, bcl-6, and K-ras, an oncogene marker and an oncogene product (see, e.g., Sensi et al., Clin Cancer Res 2006, 12:5023-32; Parmiani et al., J Immunol 2007, 178:1975-79; Novellino et al. Cancer Immunol Immunother 2005, 54:187-207).

In some embodiments, the tumor antigen is a viral antigen derived from a virus associated with a human chronic disease or cancer (such as cervical cancer). For example, in some embodiments, the viral antigen is derived from Epstein-Barr virus (EBV), HPV antigens E6 and/or E7, hepatitis C virus (HCV), hepatitis B virus (HBV), or cytomegalovirus (CMV).

Exemplary cancers or tumors and specific tumor antigens associated with such tumors (but not exclusively), include acute lymphoblastic leukemia (etv6, amil, cyclophilin b), B cell lymphoma (lg-idiotype), glioma (E-cadherin, a-catenin, b-catenin, g-catenin, p120ctn), bladder cancer (p21ras), biliary cancer (p21ras), breast cancer (MUC family, HER2/neu, c-erbB-2), cervical carcinoma (p53, p21ras), colon carcinoma (p21ras, HER2/neu, c-erbB-2, MUC family), colorectal cancer (Colorectal associated antigen (CRC)-C017-1A/GA733, APC), choriocarcinoma (CEA), epithelial cell cancer (cyclophilin b), gastric cancer (HER2/neu, c-erbB-2, ga733 glycoprotein), hepatocellular cancer (a-fetoprotein), Hodgkins lymphoma (Imp-1, EBNA-1), lung cancer (CEA, MAGE-3, NY-ESO-1), lymphoid cell-derived leukemia (cyclophilin b), melanoma (p5 protein, gp75, oncofetal antigen, GM2 and GD2 gangliosides, Melan-A/MART-1, cdc27, MAGE-3, p21ras, gplOO), myeloma (MUC family, p21ras), nonsmall cell lung carcinoma (HER2/neu, c-erbB-2), nasopharyngeal cancer (Imp-1, EBNA-1), ovarian cancer (MUC family, HER2/neu, c-erbB-2), prostate cancer (Prostate Specific Antigen (PSA) and its antigenic epitopes PSA-1, PSA-2, and PSA-3, PSMA, HER2/neu, c-erbB-2, ga733 glycoprotein), renal cancer (HER2/neu, c-erbB-2), squamous cell cancers of the cervix and esophagus, testicular cancer (NY-ESO-1), and T cell leukemia (HTLV-1 epitopes), and viral products or proteins.

In some embodiments, the CAR-expressing cell (e.g., CART cell) comprises a CAR molecule comprises an antigen binding domain directed to a B cell antigen.

In some embodiments, the B cell antigen is selected from CD10, CD19, CD20, CD22, CD34, CD123, FLT-3, ROR1, CD79b, CD179b, and CD79a.

In some embodiments, the antigen comprises a CD19 B cell antigen.

In some aspects, the portion of the CAR comprising the antigen binding domain comprises an antigen binding domain that targets CD19 or a fragment thereof. In some embodiments, the antigen binding domain targets human CD19 or a fragment thereof. In some embodiments, the antigen binding domain targets a B cell antigen (e.g., B cell surface antigen).

In some embodiments, the CD19 CAR comprises an amino acid, or has a nucleotide sequence shown in US 2015/0283178, incorporated herein by reference.

In some embodiments, the CAR molecule is capable of binding CD19 (e.g., wild-type or mutant CD19).

### Linker

Provided herein are CAR molecules that may optionally include a linker (1) between the antigen binding domain and the transmembrane domain, and/or (2) between the transmembrane domain and the cytoplasmic signaling domain. In some embodiments, the linker is a polypeptide linker.

In some embodiments, the linker is about 10 to about 250 amino acids in length, about 10 to about 200 amino acids in length, about 10 to about 175 amino acids in length, about 10 to about 150 amino acids in length, about 10 to about 125 amino acids in length, about 10 to 100 amino acids in length, about 10 to about 75 amino acids in length, about 10 to about 50 amino acids in length, about 10 to about 40 amino acids in length, about 10 to about 30 amino acids in length, about 10 to about 20 amino acids in length, or about 10 to about 15 amino acids length, and including any integer between the endpoints of any of the listed ranges. Non-limiting exemplary linkers include IgG4 hinge alone, IgG4 hinge linked to C2 and C3 domains, or IgG4 hinge linked to the CH3 domain. Further non-limiting exemplary linkers include those described in Hudecek et al., Clin. Cancer Res., 19:3153 (2013), WO 2014/031687, U.S. Pat. No. 8,822,647, and US App. No. 2014/0271635.

### Transmembrane Domain

In some embodiments, the CAR molecules described herein comprise a transmembrane domain. In some embodiments, the transmembrane domain is naturally associated with a sequence in the cytoplasmic domain. In some embodiments, the transmembrane domain is modified by one or more (e.g., two, three, four, five, six, seven, eight, nine, or ten) amino acid substitutions to avoid the binding of the domain to other transmembrane domains (e.g., the transmembrane domains of the same or different surface membrane proteins) to minimize interactions with other members of the receptor complex.

In some embodiments, the transmembrane domain is derived from a natural source. In some embodiments, the transmembrane domain is derived from any membrane-bound or transmembrane protein. Non-limiting examples of transmembrane domains for use in the present disclosure are any derived from (e.g., comprise at least the transmembrane sequence or a part of the transmembrane sequence of) the alpha, beta, or zeta chain of the T-cell receptor, CD28, CD3 epsilon, CD33, CD37, CD64, CD80, CD45, CD4, CD5, CD8, CD9, CD16, CD22, CD86, CD134, CD137 or CD154.

In some embodiments, the transmembrane domain is synthetic. For example, in some embodiments where the transmembrane domain is from a synthetic source, the transmembrane domain comprises (e.g., predominantly comprises) hydrophobic residues (e.g., leucine and valine). In some embodiments, the synthetic transmembrane domain comprises at least one (e.g., at least two, at least three, at least four, at least five, or at least six) triplet of phenylalanine, tryptophan, and valine at the end of a synthetic transmembrane domain. In some embodiments, the transmembrane domain of a CAR comprises a CD8 hinge domain.

Additional specific examples of transmembrane domains are described in the references cited herein.

### Cytoplasmic Domains

Also provided herein are CAR molecules that comprise, e.g., a cytoplasmic signaling domain that includes a cytoplasmic sequence of CD3ζ sufficient to stimulate a T cell when the antigen binding domain binds to the antigen, and optionally, a cytoplasmic sequence of one or more of co-stimulatory proteins (e.g., a cytoplasmic sequence of one or more of CD27, CD28, 4-1BB, OX40, CD30, CD40L, CD40, PD-1, PD-L1, ICOS, LFA-1, CD2, CD7, CD160, LIGHT, BTLA, TIM3, CD244, CD80, LAG3, NKG2C, B7-H3, a ligand that specifically binds to CD83, and any of the ITAM sequences described herein or known in the art) that provides for costimulation of the T cell. The stimulation of a CAR-expressing cell can result in the activation of one or more anti-cancer activities of the CAR-expressing cell. For example, in some embodiments, stimulation of a CAR-expressing cell can result in an increase in the cytolytic activity or helper activity of the CAR-expressing cell, including the secretion of cytokines. In some embodiments, the entire intracellular signaling domain of a co-stimulatory protein is included in the cytoplasmic signaling domain. In some embodiments, the cytoplasmic signaling domain includes a truncated portion of an intracellular signaling domain of a costimulatory protein (e.g., a truncated portion of the intracellular signaling domain that transduces an effector function signal in the CAR-expressing cell). Non-limiting examples of intracellular signaling domains that can be included in a cytoplasmic signaling domain include the cytoplasmic sequences of the T cell receptor (TCR) and co-receptors that act in concert to initiate signal transduction following antigen receptor engagement, as well as any variant of these sequences including at least one (e.g., one, two, three, four, five, six, seven, eight, nine, or ten) substitution and have the same or about the same functional capability.

In some embodiments, a cytoplasmic signaling domain can include two distinct classes of cytoplasmic signaling sequences: signaling sequences that initiate antigen-dependent activation through the TCR (primary cytoplasmic signaling sequences) (e.g., a CD3ζ cytoplasmic signaling sequence) and a cytoplasmic sequence of one or more of co-stimulatory proteins that act in an antigen-independent manner to provide a secondary or co-stimulatory signal (secondary cytoplasmic signaling sequences).

In some embodiments, the cytoplasmic domain of a CAR can be designed to include the CD3ζ signaling domain by itself or combined with any other desired cytoplasmic signaling sequence(s) useful in the context of a CAR. In some examples, the cytoplasmic domain of a CAR can include a CD3ζ chain portion and a costimulatory cytoplasmic signaling sequence. The costimulatory cytoplasmic signaling sequence refers to a portion of a CAR including a cytoplasmic signaling sequence of a costimulatory protein (e.g., CD27, CD28, 4-IBB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83).

In some embodiments, the cytoplasmic signaling sequences within the cytoplasmic signaling domain of a CAR are positioned in a random order. In some embodiments, the cytoplasmic signaling sequences within the cytoplasmic signaling domain of a CAR are linked to each other in a specific order. In some embodiments, a linker (e.g., any of the linkers described herein) can be used to form a linkage between different cytoplasmic signaling sequences.

In some embodiments, the cytoplasmic signaling domain is designed to include the cytoplasmic signaling sequence of CD3ζ and the cytoplasmic signaling sequence of the costimulatory protein CD28. In some embodiments, the cytoplasmic signaling domain is designed to include the cytoplasmic signaling sequence of CD3ζ and the cytoplasmic signaling sequence of costimulatory protein 4-IBB.

In some embodiments, the CAR encompasses one or more, e.g., two or more, costimulatory domains and an activation domain, e.g., primary activation domain, in the cytoplasmic portion. Exemplary CARs include intracellular components of CD3ζ, CD28, and 4-IBB. In some embodiments, the cytoplasmic signaling domain is designed to include the cytoplasmic signaling sequence of CD3ζ and the cytoplasmic signaling sequences of costimulatory proteins CD28 and 4-IBB. In some embodiments, the cytoplasmic signaling domain does not include the cytoplasmic signaling sequences of 4-IBB.

### Additional Modifications of CART Cells

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et a, Sci. Transl. Medicine, 5(215) (2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the therapeutic efficacy of CAR T cells is enhanced by modifying the CAR T cell with a nucleic acid that is capable of altering (e.g., downmodulating) expression of an endogenous gene selected from the group consisting of TCR a chain TCR b chain, beta-2 microglobulin, a HLA molecule, CTLA-4, PD1, and FAS, as described in PCT Publication WO 2016/069282 and US Publication No. 2017/0335331.

In some embodiments, the therapeutic efficacy of CAR T cells is enhanced by coexpressing in the T cells the CAR and one or more enhancers of T cell priming (“ETPs”), as described in PCT Publication WO 2015/1 12626 and US Publication No. 2016/0340406. The addition of an ETP component to the CAR T cell confers enhanced “professional” antigen presenting cell (APC) function. In some embodiments, the CAR and one or more ETPs are transiently co-expressed in the T cell. Thus, the engineered T cells are safe (given the transient nature of the CAR/ETP expression), and induce prolonged immunity via APC function.

In some embodiments, the therapeutic efficacy of CAR T cells is enhanced by coexpressing in the T cells a CAR and an inhibitory membrane protein (IMP) comprising a binding (or dimerization) domain, as described in PCT Publication WO 2016/055551 and US Publication No 2017/02921 18. The CAR and the IMP are made both reactive to a soluble compound especially through a second binding domain comprised within the CAR, thereby allowing the colocalization, by dimerization or ligand recognition, of the inhibitory signaling domain borne by the IMP and of the signal transducing domain borne by the CAR, having the effect of turning down the CAR activation. The inhibitory signaling domain is preferably the programmed death- (PD-1), which attenuates T-cell receptor (TCR)-mediated activation of IL-2 production and T cell proliferation.

In some embodiments, the therapeutic efficacy of CAR T cells is enhanced using a system where controlled variations in the conformation of the extracellular portion of a CAR containing the antigen-binding domain is obtained upon addition of small molecules as described in PCT Publication WO 2017/032777. This integrated system switches the interaction between the antigen and the antigen binding domain between on/off states. By being able to control the conformation of the extracellular portion of a CAR, downstream functions of the CAR T cell, such as cytotoxicity, can be directly modulated. Thus, a CAR can be characterized in that it comprises: a) at least one ectodomain which comprises: i) an extracellular antigen binding domain; and ii) a switch domain comprising at least a first multimerizing ligand-binding domain and a second multimerizing ligand-binding domain which are capable of binding to a predetermined multivalent ligand to form a multimer comprising said two binding domains and the multivalent ligand to which they are capable of binding: b) at least one transmembrane domain; and c) at least one endodomain comprising a signal transducing domain and optionally a co-stimulatory domain; wherein the switch domain is located between the extracellular antigen binding domain and the transmembrane domain.

### CD19 CAR Molecules

The present disclosure encompasses immune effector cells (e.g., T cells) comprising a CAR molecule that targets, e.g., specifically binds, to CD19 (CD19 CAR).

In some embodiments, the immune effector cells are engineered to express the CD19 CAR.

In some embodiments, the immune effector cells comprise a recombinant nucleic acid construct comprising nucleic acid sequences encoding the CD19 CAR.

In some embodiments, the CD19 CAR comprises an antigen binding domain that specifically binds to CD19, e.g., CD19 binding domain, a transmembrane domain, and an intracellular signaling domain.

In some embodiments, the sequence of the antigen binding domain is contiguous with and in the same reading frame as a nucleic acid sequence encoding an intracellular signaling domain.

In some aspects, CAR constructs comprise an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), and one or more intracellular stimulatory domain (e.g., an intracellular stimulatory domain described herein).

### Exemplary CD19 Antigen Binding Domains and CAR Constructs

Exemplary CD19 CAR constructs for use in the present disclosure comprise an anti-human CD19 scFv (e.g., a murine scFv or human scFv), optionally preceded with a leader sequence. The CD19 CAR construct can further include an optional hinge domain, e.g., a CD8 or CD28 hinge domain; a transmembrane domain, e.g., a CD8 or CD28 transmembrane domain; an intracellular domain, e.g., a 4-IBB or CD28 intracellular domain; and a functional signaling domain, e.g., a CD3ζ domain.

In some embodiments, the domains are contiguous with and in the same reading frame to form a single fusion protein. In some embodiments, the domains are in separate polypeptides.

In some embodiments, the CD19 CAR molecule comprises a leader sequence.

In some embodiments, the CD19 CAR molecule does not comprise a leader sequence.

In some embodiments, the CAR molecule comprises an antigen binding domain that binds specifically to CD19 (CD19 CAR).

In some embodiments, the antigen binding binds specifically to human CD19.

In some embodiments, the CD19 antibody molecule is, e.g., an antibody molecule (e.g., a humanized anti-CD19 antibody molecule) described in WO2014/153270 or WO2012/079000, each of which are incorporated herein by reference in its entirety. In some embodiments, the CD19 CAR comprises an antigen binding domain (e.g., a humanized antigen binding domain) according to Table 3 of WO2014/153270. WO2014/153270 also describes methods of assaying the binding and efficacy of various CAR constructs. In some embodiments, the scFv is a humanized variant of the scFv domain of SEQ ID NO: 12 in WO 2012/079000 which is an scFv fragment of murine origin that specifically binds to human CD19. Humanization of this mouse scFv may be desired for the clinical setting, where the mouse-specific residues may induce a human-anti-mouse antigen (HAMA) response in patients who receive CART19 treatment, e.g., treatment with T-cells transduced with the CAR 19 construct.

In some embodiments, the antigen binding domain of the CAR has the same or a similar binding specificity as the FMC63 anti-human CD19 scFv fragment described in Nicholson et al. Mol. Immun. 34 (16-17): 1157-1165 (1997). In some embodiments, the antigen binding domain of the CAR comprises the FMC63 anti-human CD19 scFv fragment described in Nicholson et al. Mol. Immun. 34 (16-17): 1157-1165 (1997).

In some embodiments, the CD19 CAR molecule comprises an anti-CD19 scFv, wherein the anti-CD19 scFv comprises an amino acid sequence having at least about 80%, about 85%, about 90%, about 91%, about 92%, about 93%, about 94%, about 95%, about 96%, about 97%, about 98%, or about 99% identity to the amino acid sequence set forth in SEQ ID NO: 1. In some embodiments, the CD19 CAR molecule comprises an anti-CD19 scFv, wherein the anti-CD19 scFv comprises the amino acid sequence set forth in SEQ ID NO: 1.

In some embodiments, the CD19 CAR molecule comprises a leader sequence. In some embodiments, the leader sequence comprises an amino acid sequence having at least about 80%, about 85%, about 90%, about 91%, about 92%, about 93%, about 94%, about 95%, about 96%, about 97%, about 98%, or about 99% identity to the amino acid sequence set forth in SEQ ID NO: 4. In some embodiments, the leader sequence comprises the amino acid sequence set forth in SEQ ID NO: 4.

In some embodiments, the CD19 CAR molecule comprises a CD8 hinge. In some embodiments, the CD8 hinge comprises an amino acid sequence having at least about 80%, about 85%, about 90%, about 91%, about 92%, about 93%, about 94%, about 95%, about 96%, about 97%, about 98%, or about 99% identity to the amino acid sequence set forth in SEQ ID NO: 5. In some embodiments, the CD8 hinge comprises the amino acid sequence set forth in SEQ ID NO: 5.

In some embodiments, the CD19 CAR molecule comprises a CD28 hinge. In some embodiments, the CD28 hinge comprises an amino acid sequence having at least about 80%, about 85%, about 90%, about 91%, about 92%, about 93%, about 94%, about 95%, about 96%, about 97%, about 98%, or about 99% identity to an amino acid sequence set forth in SEQ ID NOs: 9 or 10. In some embodiments, the CD28 hinge comprises an amino acid sequence set forth in SEQ ID NOs: 9 or 10.

In some embodiments, the CD19 CAR molecule comprises a CD8 transmembrane domain. In some embodiments, the CD8 transmembrane domain comprises an amino acid sequence having at least about 80%, about 85%, about 90%, about 91%, about 92%, about 93%, about 94%, about 95%, about 96%, about 97%, about 98%, or about 99% identity to the amino acid sequence set forth in SEQ ID NO: 6. In some embodiments, the CD8 transmembrane domain comprises the amino acid sequence set forth in SEQ ID NO: 6.

In some embodiments, the CD19 CAR molecule comprises a CD28 transmembrane domain. In some embodiments, the CD28 transmembrane domain comprises an amino acid sequence having at least about 80%, about 85%, about 90%, about 91%, about 92%, about 93%, about 94%, about 95%, about 96%, about 97%, about 98%, or about 99% identity to the amino acid sequence set forth in SEQ ID NO: 11. In some embodiments, the CD28 transmembrane domain comprises the amino acid sequence set forth in SEQ ID NO: 11.

In some embodiments, the CD19 CAR molecule comprises a 4-1BB intracellular domain. In some embodiments, the 4-1BB intracellular domain comprises an amino acid sequence having at least about 80%, about 85%, about 90%, about 91%, about 92%, about 93%, about 94%, about 95%, about 96%, about 97%, about 98%, or about 99% identity to the amino acid sequence set forth in SEQ ID NO: 7. In some embodiments, the 4-1BB intracellular domain comprises the amino acid sequence set forth in SEQ ID NO: 7.

In some embodiments, the CD19 CAR molecule comprises a CD28 intracellular domain. In some embodiments, the CD28 intracellular domain comprises an amino acid sequence having at least about 80%, about 85%, about 90%, about 91%, about 92%, about 93%, about 94%, about 95%, about 96%, about 97%, about 98%, or about 99% identity to the amino acid sequence set forth in SEQ ID NO: 12. In some embodiments, the CD28 intracellular domain comprises the amino acid sequence set forth in SEQ ID NO: 12.

In some embodiments, the CD19 CAR molecule comprises a CD3ζ signaling domain. In some embodiments, the 4 CD3ζ signaling domain comprises an amino acid sequence having at least about 80%, about 85%, about 90%, about 91%, about 92%, about 93%, about 94%, about 95%, about 96%, about 97%, about 98%, or about 99% identity to an amino acid sequence set forth in SEQ ID NOs: 8 or 13. In some embodiments, the CD3ζ signaling domain comprises an amino acid sequence set forth in SEQ ID NOs: 8 or 13.

In some embodiments, the CD19 CAR molecule comprises an anti-CD19 scFv (e.g., as set forth in SEQ ID NO: 1), a CD8 hinge (e.g., as set forth in SEQ ID NO: 5), a CD8 transmembrane domain (e.g., as set forth in SEQ ID NO: 6), a 4-1 BB co-stimulatory domain (e.g., as set forth in SEQ ID NO: 7) and a CD3ζ TCR signaling domain (e.g., as set forth in SEQ ID NOs: 8 or 13).

In some embodiments, the CD19 CAR molecule comprises an amino acid sequence having at least about 80%, about 85%, about 90%, about 91%, about 92%, about 93%, about 94%, about 95%, about 96%, about 97%, about 98%, or about 99% identity to the amino acid sequence set forth in SEQ ID NO: 2. In some embodiments, the CD19 CAR molecule comprises the amino acid sequence of SEQ ID NO: 2. In some embodiments, the CD19 CAR molecule comprises an amino acid sequence encoded by a nucleotide sequence having at least about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%/o identity to the nucleotide sequence set forth in SEQ ID NO. 3. In some embodiments, the CD19 CAR molecule comprises an amino acid sequence encoded by the nucleotide sequence set forth in SEQ ID NO: 3.

In some embodiments, the CD19 CAR molecule comprises an anti-CD19 scFv (e.g., as set forth in a SEQ ID NO: 1), a CD28 hinge (e.g., as set forth in SEQ ID NOs: 9 or 10), a CD28 transmembrane domain (e.g., as set forth in SEQ ID NO: 11), a CD28 co-stimulatory domain and a CD3ζ TCR signaling domain (e.g., as set forth in SEQ ID NOs: 8 or 13).

In some embodiments, a CD19 CAR effector cell suitable for combination with the combinations and methods disclosed herein targets CD19. In some embodiments, the CD19 CAR therapy comprises axicabtagene ciloleucel, blinatumomab, tisagenlecleucel-t, AMG-562, AUTO-1 CAR-T CD19 (Cellular Biomedicine Group), Sleeping Beauty modified CD19 CART (Ziopharm), CD19/4-1BBL armored CAR T (Juno Therapeutics), CSG-CD19, DI-B4, ET-190, GC-007F, GC-022, ICAR-19 CAR-T (Immune Cell Therapy), ICTCAR-003, iPD1 CD19 eCAR T (Marino Biotechnology), JWCAR029, PTG-01, PZ01, Sen1_1904A, Sen1_1904B, UCART-19, UWC-19, AUTO-3, BinD-19, CIK-CAR.CD19, ICTCAR-011, IM-19, JCAR-014, JCAR-017, loncastuximab tesirine, MB-CART2019.1, OXS-1550, PBCAR-0191, PCAR-019, PCAR-119, Sen1-OO1, TI-1007, XmAb-5871, inebilizumab, lisocabtagene maraleucel, XmAh-5574, 3rd generation CD19-CART cells+mbIL15 by Eden BioCell, A-329, ALLO-501, anti-CD19 (Beijing Doing Biomedical Co), anti-CD 19 CAR NK (Allife Medical Science), anti-CD 19/BCMA CAR-T (Hrain Biotechnology), ATA-2431, ATA-3219, AVA-008, CD19 dBiTE (Inovio), CD19-ATAC (Wilex), CD19/20 CAR-T (Chineo Med), CD19/CD22 dual targeting therapy (Eureka Therapeutics), CMD-502, CTX-110, CYAD-04, CYAD-221, ET-019002, FT-596, FT-819, gamma-delta CAR-T (TC Biopharm), ICTCAR-014, iDD-002, KITE-037, NI-2201, RB-1916, Sen1_002, TAC01-CD19, TC-110, TC-310, TCB-003, or TI-7007.

In some embodiments, the CD19 CAR therapy is Kymriah™ (tisagenlecleucel; Novartis; formerly CTL019; see WO 2016/109410, herein incorporated by reference in its entirety). In some embodiments, the CD19 CAR therapy is Yescarta™ (axicabtagene ciloleucel; Kite Pharma; see US 2016/0346326, herein incorporated by reference in its entirety). In some embodiments, the CD19 CAR therapy is Tecartus (Brexucabtagene autoleucel; Kite Pharma). In some embodiments, the CD19 CAR therapy is Breyanzi (lisocabtagene maraleucel, BMS).

In some embodiments, CTL019 is made by a gene modification of T-cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T-cells that are delivered to the subject on the basis of percent transgene positive T-cells.

TABLE 1
CD19 CAR Constructs

| Name/ | SEQ | |
|---|---|---|
| Identifier | ID NO | Sequence |
| CTL019 | 1 | DIQMTQTTSSLSASLGDRVTISCRASQDISKYLNWYQQKPDGTV |
| scFv domain | | KLLIYHTSRLHSGVPSRFSGSGSGTDYSLTISNLEQEDIATYFCQ |
| | | QGNTLPYTFGGGTKLEITGGGGSGGGGSGGGGSEVKLQESGPG |
| | | LVAPSQSLSVTCTVSGVSLPDYGVSWIRQPPRKGLEWLGVIWG |
| | | SETTYYNSALKSRLTIIKDNSKSQVFLKMNSLQTDDTAIYYCAK |
| | | HYYYGGSYAMDYWGQGTSVTVSS |
| CTL019 | 2 | MALPVTALLLPLALLLHAARPDIQMTQTTSSLSASLGDRVTISC |
| (amino acid) | | RASQDISKYLNWYQQKPDGTVKLLIYHTSRLHSGVPSRFSGSGS |
| | | GTDYSLTISNLEQEDIATYFCQQGNTLPYTFGGGTKLEITGGGGS |
| | | GGGGSGGGGSEVKLQESGPGLVAPSQSLSVTCTVSGVSLPDYG |
| | | VSWIRQPPRKGLEWLGVIWGSETTYYNSALKSRLTIIKDNSKSQ |
| | | VFLKMNSLQTDDTAIYYCAKHYYYGGSYAMDYWGQGTSVTV |
| | | SSTTTPAPRPPTPAPTIASQPLSLRPEACRPAAGGAVHTRGLDFA |
| | | CDIYIWAPLAGTCGVLLLSLVITLYCKRGRKKLLYIFKQPFMRP |
| | | VQTTQEEDGCSCRFPEEEEGGCELRVKFSRSADAPAYKQGQNQ |
| | | LYNELNLGRREEYDVLDKRRGRDPEMGGKPRRKNPQEGLYNE |
| | | LQKDKMAEAYSEIGMKGERRRGKGHDGLYQGLSTATKDTYD |
| | | ALHMQALPPR |
| CTL019 | 3 | ATGGCCTTACCAGTGACCGCCTTGCTCCTGCCGCTGGCCTTG |
| (nucleotide) | | CTGCTCCACGCCGCCAGGCCGGACATCCAGATGACACAGAC |
| | | TACATCCTCCCTGTCTGCCTCTCTGGGAGACAGAGTCACCAT |
| | | CAGTTGCAGGGCAAGTCAGGACATTAGTAAATATTTAAATTG |
| | | GTATCAGCAGAAACCAGATGGAACTGTTAAACTCCTGATCTA |
| | | CCATACATCAAGATTACACTCAGGAGTCCCATCAAGGTTCAG |
| | | TGGCAGTGGGTCTGGAACAGATTATTCTCTCACCATTAGCAA |
| | | CCTGGAGCAAGAAGATATTGCCACTTACTTTTGCCAACAGGG |
| | | TAATACGCTTCCGTACACGTTCGGAGGGGGGACCAAGCTGG |
| | | AGATCACAGGTGGCGGTGGCTCGGGCGGTGGTGGGTCGGGT |
| | | GGCGGCGGATCTGAGGTGAAACTGCAGGAGTCAGGACCTGG |
| | | CCTGGTGGCGCCCTCACAGAGCCTGTCCGTCACATGCACTGT |
| | | CTCAGGGGTCTCATTACCCGACTATGGTGTAAGCTGGATTCG |
| | | CCAGCCTCCACGAAAGGGTCTGGAGTGGCTGGGAGTAATAT |
| | | GGGGTAGTGAAACCACATACTATAATTCAGCTCTCAAATCCA |
| | | GACTGACCATCATCAAGGACAACTCCAAGAGCCAAGTTTTCT |
| | | TAAAAATGAACAGTCTGCAAACTGATGACACAGCCATTTACT |
| | | ACTGTGCCAAACATTATTACTACGGTGGTAGCTATGCTATGG |
| | | ACTACTGGGGCCAAGGAACCTCAGTCACCGTCTCCTCAACCA |
| | | CGACGCCAGCGCCGCGACCACCAACACCGGCGCCCACCATC |
| | | GCGTCGCAGCCCCTGTCCCTGCGCCCAGAGGCGTGCCGGCC |
| | | AGCGGGGGGGGCGCAGTGCACACGAGGGGGCTGGACTTCG |
| | | CCTGTGATATCTACATCTGGGCGCCCTTGGCCGGGACTTGTG |
| | | GGGTCCTTCTCCTGTCACTGGTTATCACCCTTTACTGCAAACG |
| | | GGGCAGAAAGAAACTCCTGTATATATTCAAACAACCATTTAT |
| | | GAGACCAGTACAAACTACTCAAGAGGAAGATGGCTGTAGCT |
| | | GCCGATTTCCAGAAGAAGAAGAAGGAGGATGTGAACTGAGA |
| | | GTGAAGTTCAGCAGGAGCGCAGACGCCCCCGCGTACAAGCA |
| | | GGGCCAGAACCAGCTCTATAACGAGCTCAATCTAGGACGAA |
| | | GAGAGGAGTACGATGTTTTGGACAAGAGACGTGGCCGGGAC |
| | | CCTGAGATGGGGGGAAAGCCGAGAAGGAAGAACCCTCAGG |
| | | AAGGCCTGTACAATGAACTGCAGAAAGATAAGATGGCGGAG |
| | | GCCTACAGTGAGATTGGGATGAAAGGCGAGCGCCGGAGGGG |
| | | CAAGGGGCACGATGGCCTTTACCAGGGTCTCAGTACAGCCA |
| | | CCAAGGACACCTACGACGCCCTTCACATGCAGGCCCTGCCCC |
| | | CTCGC |
| Leader | 4 | MALPVTALLLPLALLLHAARP |
| sequence | | |
| CD8 hinge | 5 | TTTPAPRPPTPAPTIASQPLSLRPEACRPAAGGAVHTRGLDFACD |
| CD8 | 6 | IYIWAPLAGTCGVLLLSLVITLYC |
| transmembrane | | |
| 4-1BB | 7 | KRGRKKLLYIFKQPFMRPVQTTQEEDGCSCRFPEEEEGGCEL |
| intracellular | | |
| domain | | |
| CD3ζ | 8 | RVKFSRSADAPAYKQGQNQLYNELNLGRREEYDVLDKRRGRD |
| domain | | PEMGGKPRRKNPQEGLYNELQKDKMAEAYSEIGMKGERRRGK |
| variant 1 | | GHDGLYQGLSTATKDTYDALHMQALPPR |
| CD28 hinge | 9 | LDNEKSNGTIIHVKGKHLCPSPLFPGPSKPFWVLVVVGGVLACY |
| variant 1 | | SLLVTVAFIIFWVRSKRSRLLHSDYMNMTPRRPGPTRKHYQPY |
| | | APPRDFAAYRS |
| CD28 hinge | 10 | IEVMYPPPYLDNEKSNGTIIHVKGKHLCPSPLFPGPSKP |
| variant 2 | | |
| CD28 | 11 | FWVLVVVGGVLACYSLLVTVAFIFWV |
| transmembrane | | |
| CD28 | 12 | RSKRSRLLHSDYMNMTPRRPGPTRKHYQPYAPPRDFAAYRS |
| signaling | | |
| domain | | |
| CD3ζ | 13 | RVKFSRSADAPAYQQGQNQLYNELNLGRREEYDVLDKRRGRD |
| domain | | PEMGGKPRRKNPQEGLYNELQKDKMAEAYSEIGMKGERRRGK |
| variant 2 | | GHDGLYQGLSTATKDTYDALHMQALPPR |

### CAR-Expressing Cells

In some embodiments, the cell expressing the CAR molecule (also referred to herein as a “CAR-expressing cell”) is a cell or population of cells as described herein, e.g., a human immune effector cell or population of cells (e.g., a human T-cell). The cell or population of cells are genetically engineered to express the CAR molecule, e.g., by introducing a nucleic acid molecule encoding the CAR molecule into a composition comprising the cell or population of cells.

In some embodiments, CAR-expressing cells (e.g. CAR-T cells) are cells that are derived from a patient with a disease or condition and genetically modified in vitro to express at least one CAR with an arbitrary specificity to a target antigen. In some embodiments, the cells perform at least one effector function (e.g. induction of cytokines) that is stimulated or induced by the specific binding of the ligand to the CAR and that is useful for treatment of the same patient's disease or condition. In some embodiments, the effector cells are T cells (e.g. CD8 T cells and/or CD4 T cells). One skilled in the art would understand that other cell types (e.g. an NK cell, a macrophage, a hematopoietic stem or progenitor cell) may be engineered to express CARs and that a CAR-expressing cell may comprise an effector cell other than a T cell. In some embodiments, the effector cell is a T cell (e.g. a CD8 T cell) that exerts its effector function (e.g. a cytotoxic T cell response) on a target cell when brought in contact or in proximity to the target or target cell (e.g. a cancer cell) (see e.g., Chang and Chen (2017) Trends Mol Med 23(5):430-450).

In some embodiments, the CAR-expressing cell is an autologous CAR-expressing cell (e.g., an autologous CAR-expressing T cell). In some embodiments, the CAR cell therapy (e.g., CART cell therapy) is carried out by autologous transfer, in which the immune cells are isolated and/or otherwise prepared from the subject who is to receive the cell therapy, or from a sample derived from such a subject. Thus, in some aspects, the immune cells are derived from a subject, e.g., patient, in need of a treatment and the cells, following isolation and processing are administered to the same subject.

In some embodiments, the CAR-expressing cell is an allogeneic CAR-expressing cell (e.g., a CAR-expressing T-cell). In some embodiments, the CAR cell therapy (e.g., CART cell therapy) is carried out by allogeneic transfer, in which the immune cells are isolated and/or otherwise prepared from a subject other than a subject who is to receive or who ultimately receives the cell therapy, e.g., a first subject. In such embodiments, the immune cells are then administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the cell expressing the CAR molecule is a CD19 CAR T-cell (CART19).

Prolonged exposure of T cells to their cognate antigen can result in exhaustion of effector functions, enabling the persistence of infected or transformed cells. Recently developed strategies to stimulate or rejuvenate host effector function using agents that induce an immune checkpoint blockade have resulted in success towards the treatment of several cancers. Emerging evidence suggests that T cell exhaustion may also represent a significant impediment in sustaining long-lived antitumor activity by CAR-expressing T cells. In some embodiments, the differentiation status of the patient-harvested T cells prior to CAR transduction and the conditioning regimen a patient undergoes before reintroducing the CAR-T cells (e.g., addition or exclusion of alkylating agents, fludarabine, total-body irradiation) can profoundly affect the persistence and cytotoxic potential of CAR-T cells. In vitro culture conditions that stimulate (via anti-CD3/CD28 or stimulator cells) and expand (via cytokines, such as IL-2) T cell populations can also alter the differentiation status and effector function of CAR-T cells (Ghoneim et al., (2016) Trends in Molecular Medicine 22(12): 1000-1011).

### Vectors

In some embodiments, the cell expressing the CAR molecule comprises a vector that includes a nucleic acid sequence encoding the CAR molecule. Such vectors include viral and non-viral systems. In some embodiments, the vector is selected from the group consisting of a DNA, a RNA, a plasmid, a lentivirus vector, adenoviral vector, or a retrovirus vector. In some embodiments, the vector is a lentivirus vector.

In some embodiments, the vector further comprises a promoter.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In some embodiments, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1.5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109. [0465]

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, the vector is an in vitro transcribed vector, e.g., a vector that transcribes RNA of a nucleic acid molecule described herein.

### Methods and Compositions for Producing CAR-Expressing Cells

The present disclosure also provides, in certain aspects, a method of making a population of immune effector cells (e.g., T-cells or NK cells) that can be engineered to express a CAR (e.g., a CAR described herein), the method comprising: providing a population of immune effector cells; and contacting, e.g., transducing, the immune effector cells with a nucleic acid encoding a CAR molecule. In some embodiments, the method comprises contacting the immune effector cells with a BTK inhibitor (e.g., vecabrutinib) under conditions sufficient to inhibit a target of the kinase inhibitor.

In some embodiments, a subject's effectors cells (e.g., T cells) are genetically modified with a CAR (Sadelain et al., Cancer Discov. 3:388-398, 2013). For example, an effector cell (e.g., T cell) is provided and a recombinant nucleic acid encoding a chimeric antigen receptor is introduced into the patient-derived effector cell (e.g., T cell) to generate a CAR cell. In some embodiments, effector cells (e.g., T cells) not derived from the subject are genetically modified with a CAR. For example, in some embodiments, effector cells (e.g., T cells) are allogeneic cells that have been engineered to be used as an “off the shelf adoptive cell therapy, such as Universal Chimeric Antigen Receptor T cells (UCARTs). UCARTs are allogeneic CAR T cells that have been engineered to be used for treating the largest number of patients with a particular cancer type. Non-limiting examples of UCARTs under development include those that target the following tumor antigens: CD19, CD123, CD22, CS1 and CD38.

A variety of different methods known in the art can be used to introduce any of the nucleic acids or expression vectors disclosed herein into an effector cell (e.g., T cell). Nonlimiting examples of methods for introducing nucleic acid into a an effector cell (e.g., T cell) include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection. Furthermore, the CRISPR/Cas9 genome editing technology known in the art can be used to introduce CAR nucleic acids into effector cells (e.g., T cells) and/or to introduce other genetic modifications (e.g., as described below) into effector cells (e.g., T cells) to enhance CAR cell activity (for use of CRISPR/Cas9 technology in connection with CAR T cells, see e.g., U.S. Pat. Nos. 9,890,393; 9,855,297; US 2017/0175128; US 2016/0184362; US 2016/0272999; WO 2015/161276; WO 2014/191128; CN 106755088; CN 106591363; CN 106480097; CN 106399375; CN 104894068).

In some aspects, the disclosure provides a method of making a CAR-expressing cell (e.g., a CAR-expressing immune effector cell or population of cells), comprising: introducing (e.g., transducing) a nucleic acid encoding a CAR molecule into the cell or population of cells under conditions such that the CAR molecule is expressed.

In some aspects, the disclosure provides a method of making a CAR-expressing cell (e.g., a CAR-expressing immune effector cell or population of cells), comprising: contacting the cell or population of cells with a BTK inhibitor (e.g., vecabrutinib); and introducing (e.g., transducing) a nucleic acid encoding a CAR molecule into the cell or population of cells under conditions such that the CAR molecule is expressed.

In some embodiments, the cell or population of cells is incubated or cultured in the presence of a BTK inhibitor described herein (e.g., vecabrutinib). In some embodiments, the incubation or culturing is performed prior to, during, or subsequent to gene transfer, e.g., via transducing a nucleic acid sequence encoding a CAR molecule (e.g., a CD19 CAR molecule) under conditions such that the CAR molecule is expressed. In some embodiments, the BTK inhibitor (e.g., vecabrutinib) is added during the cell manufacturing process (e.g., during the process of engineering CAR-T cells). In some embodiments, the presence of the BTK inhibitor improves proliferation and/or effector function of the cell or population of cells produced. In some embodiments, the presence of the BTK inhibitor (e.g., vecabrutinib) provides a population of cells with an increased number of CAR-expressing cells (e.g., CAR-expressing T cells). In some embodiments, the presence of the BTK inhibitor (e.g., vecabrutinib) provides a population of cells comprising CAR-expressing cells (e.g., CAR-expressing T cells) having a surface phenotype associated with a less-differentiated, less-activated, and/or less-exhausted phenotypic state, despite substantial expansion and/or proliferation (e.g., as determined by expression of cell surface markers measured by flow cytometry).

In some embodiments of the methods of producing CAR-expressing cells, the CAR molecule encoded by the nucleic acid is a CAR molecule that binds an antigen described herein, e.g., CD19. In some embodiments, the method further comprises culturing the cell or cells under conditions that allow the cell or at least a sub-population of the cells to express the CAR molecule. In some embodiments, the cell is a T-cell or NK cell, or the population of cells includes T-cells, NK cells, or both.

In some embodiments, the cells, e.g., T cells, may be transfected either during or after expansion e.g., with a CAR molecule described herein. The genetically modified cell population can then be liberated from the initial stimulus (the CD3/CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g., natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some embodiments, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

In some embodiments, the method comprises contacting the cell or cells with a BTK inhibitor described herein (e.g., vecabrutinib).

In some embodiments, the BTK inhibitor is added after the cell or cells are harvested or before the cell or cells are stimulated.

In some aspects, the present disclosure also provides a reaction mixture comprising the BTK inhibitor (e.g., vecabrutinib) and a CAR molecule or a nucleic acid encoding a CAR molecule. In some embodiments, the reaction mixture further comprises a population of immune effector cells.

In some embodiments, one or more of the immune effector cells expresses the CAR molecule or comprises the nucleic acid encoding the CAR molecule.

In some embodiments, the reaction mixture comprises cancer cells.

In some embodiments, the cancer cells are hematological cancer cells.

### Compositions

In some aspects, the present disclosure provides a composition comprising a cell expressing a CAR (e.g., CD19 CAR) and a BTK inhibitor described herein (e.g., vecabrutinib). The CAR-expressing cell and the inhibitor may be in the same or different formulation or pharmaceutical composition.

In some embodiments, the CAR-expressing cell and the inhibitor may be in the same formulation or pharmaceutical composition.

In some embodiments, the CAR-expressing cell and the BTK inhibitor may be in different formulation or pharmaceutical composition.

Pharmaceutical compositions of the present disclosure may comprise a CAR-expressing cell, e.g., a plurality of CAR-expressing cells, in combination with one or more pharmaceutically or physiologically acceptable carriers, diluents or excipients.

In some embodiments, the composition comprises buffers such as neutral buffered saline, or phosphate buffered saline; carbohydrates such as glucose, mannose, sucrose, dextrans, or mannitol; proteins; polypeptides or amino acids such as glycine; antioxidants; chelating agents such as EDTA or glutathione; adjuvants (e.g., aluminum hydroxide); and preservatives. In some embodiments, compositions of the present disclosure are formulated for intravenous administration.

### Biological Assays

Combinations designed, selected and/or optimized by methods described above, once produced, can be characterized using a variety of assays known to those skilled in the art to determine whether the peptides have biological activity. For example, the combination can be characterized by conventional assays, including but not limited to those assays described below, to determine whether they have a predicted activity, binding activity and/or binding specificity.

Various in vitro or in vivo biological assays may be suitable for detecting the effect of the peptides of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, a parameter associated with administering a combination therapy described herein, is or includes assessment of the exposure, persistence and proliferation of the CAR-expressing cells, e.g., CAR-expressing T cells, prior to or subsequent to administration to a subject. In some embodiments, the increased exposure, or prolonged expansion and/or persistence of the cells, and/or changes in cell phenotypes or functional activity of the CAR-expressing cells can be measured by assessing the characteristics of the cells in vitro (e.g., prior to administration) or ex vivo (e.g., subsequent to administration). In some embodiments, such assays can be used to determine or confirm the function of the CAR-expressing cells used for the CAR therapy (e.g., CART cell therapy), before or after administering the combination therapy provided herein.

In some embodiments, the administration of the BTK inhibitor (e.g., vecabrutinib) is designed to promote exposure of the subject to the CAR-expressing cells (e.g., CART cells), such as by promoting their expansion and/or persistence over time. In some embodiments, the CAR therapy (e.g., CART therapy) exhibits increased or prolonged expansion and/or persistence in the subject as compared to a method in which the CAR therapy is administered to the subject in the absence of the BTK inhibitor (e.g., vecabrutinib).

Provides herein are methods for detecting the presence and/or amount of CAR-expressing cells (e.g., CAR-expressing T cells) in the subject following administration of the CAR therapy (e.g., CART therapy) and before, during and/or after the administration of the BTK inhibitor (e.g., vecabrutinib). Methods for determining the presence and/or amount of CAR-expressing cells (e.g., CAR-expressing T cells) comprise obtaining a sample (e.g., blood or tissue sample) from subjects that have been administered the CAR therapy (e.g., CART therapy), and determining the number or ratio of the CAR-expressing cells (e.g., CAR-expressing T cells) in the sample. In some embodiments, quantitative PCR (qPCR) is used to assess the quantity of CAR-expressing cells in the sample. In some embodiments, persistence is quantified as copies of DNA or plasmid encoding the CAR per microgram of DNA. In some embodiments, flow cytometry is used to assess the quantity of CAR-expressing cells in the sample. In some embodiments, persistence is quantified as the number of CAR-expressing, cells per microliter of the sample (e.g., per microliter of serum), per microgram of the sample (e.g., per microgram of tissue), or per total number of peripheral blood mononuclear cells (PBMCs), immune cells (e.g., CD45-expressing immune cells), or T cells in the sample.

Approaches for selecting and/or isolating cells, e.g., using flow cytometry, may include use of CAR-specific antibodies (e.g., Brentjens et al., Sci. Transl. Med. 2013 March; 5(177): 177ra38) Protein L (Zheng et al., J. Transl. Med. 2012 February; 10:29), epitope tags, such as Strep-Tag sequences, introduced directly into specific sites in the CAR, whereby binding reagents for Strep-Tag are used to expansion and/or persistence of the cells. directly assess the CAR (Liu et al. (2016) Nature Biotechnology, 34:430; WO2015095895) and monoclonal antibodies that specifically bind to a CAR polypeptide (see WO2014190273).

In some embodiments, a parameter associated with administering a combination therapy described herein, is or includes assessment of the functional activity of the CAR-expressing cells, e.g., CAR-expressing T cells, prior to or subsequent to administration to a subject. In some embodiments, any of the known assays in the art for assessing the activity, phenotypes, proliferation and/or function of immune cells can be used. Parameters to assess include specific binding of a CAR-expressing cell (e.g., CAR-expressing T cell) to an antigen in vivo (e.g., by imaging), or ex vivo (e.g., by ELISA or flow cytometry). In some embodiments, the ability of the CAR-expressing cells (e.g., CAR-expressing T cells) to destroy target cells is measured using any suitable method known in the art, such as cytotoxicity assays described in, for example, Kochenderfer et al, J. Immunotherapy, 32(7): 689-702 (2009), and Herman et al, J. Immunological Methods, 285(1): 25-40 (2004). In some embodiments, the biological activity of the CAR-expressing cells (e.g., CAR-expressing T cells) is measured by assaying expression and/or secretion of one or more cytokines, such as CD107a, IFNy, IL-2, GM-CSF and TNFa, and/or by assessing cytolytic activity following exposure to target antigen.

In some embodiments, assays for the activity, phenotypes, proliferation and/or function of CAR-expressing T cells, include, but are not limited to, ELISPOT, ELISA, cellular proliferation, cytotoxic lymphocyte (CTL) assay, binding to the T cell epitope, antigen or ligand, or intracellular cytokine staining, proliferation assays, lymphokine secretion assays, direct cytotoxicity assays, and limiting dilution assays. In some embodiments, proliferative responses of the T cells can be measured, e.g. by incorporation of H-thymidine, BrdU (5-Bromo-2′-Deoxyuridine) or 2′-deoxy-5-ethynyluridine (EdU) into their DNA or dye dilution assays, using dyes such as carboxyfluorescein diacetate succinimidyl ester (CFSE), CellTrace Violet, or membrane dye PKH26. In some embodiments, the assays comprise measuring cytokine and/or chemokine production by the CAR-expressing T cells in vitro following exposure to target antigen, e.g., using a multiplex cytokine assay or ELISA. In some embodiments, the assays comprise measuring cytokines and/or chemokines present in a tissue sample (e.g., blood sample) obtained from a subject administered the CAR-expressing T cells, e.g., using a multiplex cytokine assay or ELISA

In some embodiments, the biological assay is described in the Examples herein.

### Method of Use

In some aspects, the present disclosure provides a combination comprising:
a. immune effector cells (e.g., T-cells or NK cells) engineered to express a Chimeric Antigen Receptor (CAR); and
b. a BTK inhibitor (e.g., vecabrutinib).

In some aspects, the present disclosure provides a combination comprising:
a. CD19 CAR T-cells; and
b. vecabrutinib.

In some embodiments, vecabrutinib is the succinic acid salt.

In some aspects, the present disclosure provides compositions and methods for treating a disease associated with expression of an antigen (e.g., CD19) or condition associated with cells which express the antigen (e.g., CD19).

A CAR-expressing cell described herein may be used in combination with a BTK inhibitor.

In some embodiments, the combination of the CAR-expressing cell and the BTK inhibitor can be used in further combination with other known agents and therapies.

In some embodiments, the CAR therapy and the BTK inhibitor (e.g., vecabrutinib) is administered in an amount or dose that is higher, lower or the same than the amount or dosage of each agent used individually, e.g., as a monotherapy.

In some embodiments, compositions will be formulated as a combination therapeutic or administered separately.

In some embodiments, the present disclosure provides kits that include one or more compound of the present disclosure.

In some embodiments, a BTK inhibitor, is used in combination with a CAR-expressing cell to treat a disease or disorder.

In some embodiments, the disease or disorder is a cancer.

In some embodiments, the cancer is a hematological cancer or a solid cancer.

In some embodiments, the CAR-expressing cell is a CD19 CAR T-cell.

In some embodiments, the BTK inhibitor is vecabrutinib.

In some aspects, the present disclosure provides a method of treating or preventing a disease or disorder disclosed herein in a subject in need thereof, comprising administering to the subject a combination of the present disclosure, or a pharmaceutical composition of the present disclosure.

In some aspects, the present disclosure provides a method of treating a disease or disorder disclosed herein in a subject in need thereof, comprising administering to the subject a combination of the present disclosure, or a pharmaceutical composition of the present disclosure.

In some aspects, the present disclosure provides a method of treating or preventing a cancer in a subject in need thereof, comprising administering to the subject a combination of the present disclosure, or a pharmaceutical composition of the present disclosure.

In some aspects, the present disclosure provides a method of treating a cancer in a subject in need thereof, comprising administering to the subject a combination of the present disclosure, or a pharmaceutical composition of the present disclosure.

In some aspects, the present disclosure provides a method of treating or preventing a hematological cancer in a subject in need thereof, comprising administering to the subject a combination of the present disclosure, or a pharmaceutical composition of the present disclosure.

In some aspects, the present disclosure provides a method of treating a hematological cancer in a subject in need thereof, comprising administering to the subject a combination of the present disclosure, or a pharmaceutical composition of the present disclosure.

In some aspects, the present disclosure provides a method of treating or preventing a disease or disorder disclosed herein in a subject in need thereof, comprising administering to the subject a therapeutically effective amount of a combination of the present disclosure, or a pharmaceutical composition of the present disclosure.

In some aspects, the present disclosure provides a method of treating a disease or disorder disclosed herein in a subject in need thereof, comprising administering to the subject a therapeutically effective amount of a combination of the present disclosure, or a pharmaceutical composition of the present disclosure.

In some aspects, the present disclosure provides a method of treating or preventing a cancer in a subject in need thereof, comprising administering to the subject a therapeutically effective amount of a combination of the present disclosure, or a pharmaceutical composition of the present disclosure.

In some aspects, the present disclosure provides a method of treating a cancer in a subject in need thereof, comprising administering to the subject a therapeutically effective amount of a combination of the present disclosure, or a pharmaceutical composition of the present disclosure.

In some aspects, the present disclosure provides a method of treating or preventing a hematological cancer in a subject in need thereof, comprising administering to the subject a therapeutically effective amount of a combination of the present disclosure, or a pharmaceutical composition of the present disclosure.

In some aspects, the present disclosure provides a method of treating a hematological cancer in a subject in need thereof, comprising administering to the subject a therapeutically effective amount of a combination of the present disclosure, or a pharmaceutical composition of the present disclosure.

In some aspects, the present disclosure provides a combination of the present disclosure for use in treating or preventing a disease or disorder disclosed herein.

In some aspects, the present disclosure provides a combination of the present disclosure for use in treating a disease or disorder disclosed herein.

In some aspects, the present disclosure provides a combination of the present disclosure for use in treating or preventing a cancer.

In some aspects, the present disclosure provides a combination of the present disclosure for use in treating a cancer.

In some aspects, the present disclosure provides a combination of the present disclosure for use in treating or preventing a hematological cancer.

In some aspects, the present disclosure provides a combination of the present disclosure for use in treating a hematological cancer.

In some aspects, the present disclosure provides use of a combination of the present disclosure in the manufacture of a medicament for treating or preventing a disease or disorder disclosed herein.

In some aspects, the present disclosure provides use of a combination of the present disclosure in the manufacture of a medicament for treating a disease or disorder disclosed herein.

In some aspects, the present disclosure provides use of a combination of the present disclosure in the manufacture of a medicament for treating or preventing a cancer.

In some aspects, the present disclosure provides use of a combination of the present disclosure in the manufacture of a medicament for treating a cancer.

In some aspects, the present disclosure provides use of a combination of the present disclosure in the manufacture of a medicament for treating or preventing a hematological cancer.

In some aspects, the present disclosure provides use of a combination of the present disclosure in the manufacture of a medicament for treating a hematological cancer.

In some embodiments, the hematological cancer is a leukemia, a lymphoma, or a malignant lymphoproliferative conditions that affect blood, bone marrow and/or the lymphatic system.

In some embodiments, the hematologic cancer is a leukemia.

In some embodiments, the cancer is an acute leukemia.

In some embodiments, the hematological cancer is chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia and myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, or unclassifiable lymphoma.

In some embodiments, the acute leukemia is B-cell acute lymphoid leukemia (BALL), T-cell acute lymphoid leukemia (TALL), small lymphocytic leukemia (SLL), acute lymphoid leukemia (ALL), chronic myelogenous leukemia (CML), chronic lymphocytic leukemia (CLL). In some embodiments, the hematologic cancer is mantle cell lymphoma (MCL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, or Waldenstrom macroglobulinemia.

In some embodiments, the leukemia is acute leukemia or chronic leukemia. In some embodiments, acute leukemia is acute myelogenous leukemia (AML) or acute lymphoid leukemia (ALL). In some embodiments, the chronic leukemia is chronic myelogenous leukemia (CML) or chronic lymphoid leukemia (CLL).

In some embodiments, the lymphoma is non-Hodgkin lymphoma or Hodgkin lymphoma.

In some embodiments, the Non-Hodgkin lymphoma (NHL) is a B-cell non-Hodgkin lymphoma. In some embodiments, the B-cell non-Hodgkin lymphoma is Burkitt lymphoma, chronic lymphocytic leukemia/small lymphocytic lymphoma (CLL/SLL), diffuse large B-cell lymphoma (DLBCL), follicular lymphoma, immunoblastic large cell lymphoma, precursor B-lymphoblastic lymphoma, or mantle cell lymphoma.

In some embodiments, the T-cell non-Hodgkin lymphoma is mycosis fungoides, anaplastic large cell lymphoma, or precursor T-lymphoblastic lymphoma.

In some embodiments, the cancer is a solid cancer.

In some embodiments, solid cancers include, but are not limited to, uterine cancer, colon cancer, ovarian cancer, rectal cancer, skin cancer, stomach cancer, lung cancer, non-small cell carcinoma of the lung, breast cancer, cancer of the small intestine, testicular cancer, cancer of the anal region, cancer of the endocrine system, cancer of the thyroid gland, cancer of the parathyroid gland, rectal cancer, renal-cell carcinoma, liver cancer, cancer of the esophagus, melanoma, cutaneous or intraocular malignant melanoma, uterine cancer, brain cancer, brain stem glioma, pituitary adenoma, Kaposi's sarcoma, cancer of the adrenal gland, bone cancer, pancreatic cancer, cancer of the head or neck, epidermoid cancer, carcinoma of the endometrium, carcinoma of the vagina, cervical cancer, sarcoma, uterine cancer, stomach cancer, esophageal cancer, colorectal cancer, liver cancer, prostate cancer, carcinoma of the cervix squamous cell cancer, carcinoma of the fallopian tubes, sarcoma of soft tissue, cancer of the urethra, carcinoma of the vulva, cancer of the kidney or ureter, carcinoma of the renal pelvis, spinal axis tumor, cancer of the penis, cancer of the bladder, neoplasm of the central nervous system (CNS), primary CNS lymphoma, tumor angiogenesis, metastatic lesions of said cancers, and/or combinations thereof.

In some embodiments, the disease or disorder is cytokine release syndrome (CRS).

In some embodiments, the disease or disorder is a symptom of cytokine release syndrome (CRS).

In some embodiments, symptoms of CRS can include, but are not limited to, neurologic toxicity, disseminated intravascular coagulation, cardiac dysfunction, adult respiratory distress syndrome, renal failure, and/or hepatic failure. For example, symptoms of CRS can include fever with or without rigors, fatigue, malaise, myalgias, vomiting, headache, nausea, anorexia, arthralgias, diarrhea, rash, hypoxemia, tachypnea, hypotension, widened pulse pressure, potentially diminished cardiac output (late), increased cardiac output (early), azotemia, hypofibrinogenemia with or without bleeding, elevated D-dimer, hyperbilirubinemia, transaminitis, confusion, delirium, mental status changes, hallucinations, tremor, seizures, altered gait, word finding difficulty, frank aphasia, or dysmetria.

In some aspects, the present disclosure provides methods for treating CLL.

In some embodiments, CLL is a hematologic malignancy characterized by a progressive accumulation of clonally-derived B cells, e.g. CD19+, in the blood, bone marrow and lymphatic tissue. Although considered the same disease as CLL, in some cases, SLL is used to refer to the disease when characterized by lymphadenopathy (cancer cells found in the lymph nodes), whereas in CLL cancer cells are found mostly in the blood and bone marrow. Patients with progressive CLL generally have a poor prognosis with an overall survival (OS) of less than 1 year as reported in some studies (Jain et al. (2016) Expt. Rev. Hematol., 9:793-801).

Ibrutinib is a current first-line approved therapy for CLL patients. Although partial responses (PRs) can be sustained for a long duration, studies found that around 25% of previously treated CLL patients discontinue ibrutinib (Jain et al. (2015) Blood, 125:2062-2067; Maddocks (2015) JAMA Oncol., 1:80-87; Jain et al. (2017) Cancer, 123:2268-2273). In some cases, discontinuation of ibrutinib is due to progression of CLL or Richter's transformation. Further, mutations in BTK or the downstream effector phospholipase Cy2 (PLCy2) can emerge during ibrutinib treatment and are associated with ibrutinib resistance and ultimately relapse (Woyach et al. (2014) N. Engl. J. Med., 370:2286-2294). Such mutations are observed in 87% of CLL patients relapsing on ibrutinib.

Accordingly, the disclosure provides methods for treating a hematological malignancy (e.g., CLL/SLL) in a subject that is refractory to and/or develops resistance following the course of treatment with ibrutinib comprising administering a BTK inhibitor described herein (e.g., vecabrutinib) and a CART therapy (e.g., CART19 therapy). In some embodiments, the subject undergoes relapse following remission resulting from treatment with ibrutinib or has not achieved a complete response for a substantial period (e.g., a period of 6-12 months) following treatment with ibrutinib. In some embodiments, the subject is one that is not responsive to and/or has been deemed refractory to or resistant to treatment with ibrutinib.

In some embodiments, disclosure provides methods for treating a cancer in a subject, wherein the cancer comprises one or more mutations in BTK relative to wild-type BTK that render it resistant to ibrutinib, the method comprising administering a BTK inhibitor described herein (e.g., vecabrutinib) and a CART therapy (e.g., CART19 therapy).

In some embodiments, the one or more mutations is selected from BTK^{C481S} and BTK^{C481R}.

In some embodiments, the disclosure provides methods for improving the function of CAR-expressing cells in vivo in a subject, comprising administering the CAR-expressing cells in combination with a BTK inhibitor described herein (e.g., vecabrutinib).

In some embodiments, the disclosure provides a method for increasing the expansion of CAR-expressing cells (e.g., CART19 cells) in vivo in a subject, the method comprising administering the CAR-expressing cells in combination with a BTK inhibitor described herein (e.g., vecabrutinib).

In some embodiments, the disclosure provides a method for increasing the proliferation of CAR-expressing cells (e.g., CART19 cells) in vivo in a subject, the method comprising administering the CAR-expressing cells in combination with a BTK inhibitor described herein (e.g., vecabrutinib).

In some embodiments, the disclosure provides a method for reducing immune suppression of CAR-expressing cells (e.g., CART19 cells) in vivo in a subject, the method comprising administering the CAR-expressing cells in combination with a BTK inhibitor described herein (e.g., vecabrutinib).

In some embodiments, the method comprises administering the BTK inhibitor (e.g., vecabrutinib) simultaneously with the administration of the CAR-expressing cells (e.g., CART cells). In some embodiments, the method comprises administering the BTK inhibitor (e.g., vecabrutinib) following the administration of the CAR-expressing cells (e.g., CART cells). In some embodiments, the method comprises sequential or intermittent administration of the BTK inhibitor (e.g., vecabrutinib) and/or the CAR-expressing cells (e.g., CART cells).

In some embodiments, the subject is administered the CAR-expressing cells and the BTK inhibitor (e.g., vecabrutinib) is administered simultaneously or sequentially.

In some aspects, the present disclosure provides a method for treating or preventing a disease or disorder (e.g., cancer) in a subject, comprising administering to the subject a population of CAR-expressing cells, or pharmaceutical composition thereof, wherein the CAR-expressing cells are contacted ex vivo with the BTK inhibitor (e.g., vecabrutinib) prior to the administration.

In some aspects, the present disclosure provides a method for treating a disease or disorder (e.g., cancer) in a subject, comprising administering to the subject a population of CAR-expressing cells, or pharmaceutical composition thereof, wherein the CAR-expressing cells are contacted ex vivo with the BTK inhibitor (e.g., vecabrutinib) prior to the administration.

In some embodiments, the population of CAR-expressing immune effector cells is prepared by harvesting a population of cells from a subject (e.g., a population of cells comprising white blood cells), selecting and activating a population of cells enriched in immune effector cells (e.g., T cells), transducing the population of cells ex vivo with a nucleic acid encoding a CAR molecule described herein (e.g., a CD19 CAR molecule) to generate a population of CAR-expressing immune effector cells (e.g., CAR-expressing T cells), and administering the population of CAR-expressing immune effector cells, or pharmaceutical composition thereof, to a subject having the disease or disorder (e.g., cancer).

In some embodiments, the population of immune effector cells is contacted ex vivo with a BTK inhibitor described herein (e.g., vecabrutinib) prior to, during, or subsequent to the transducing to improve or enhance proliferation of the CAR-expressing cells. In some embodiments, the population of CAR-expressing cells is contacted ex vivo with the BTK inhibitor (e.g., vecabrutinib) prior to, during, or subsequent to the transducing to improve or enhance effector function of the population of CAR-expression cells. In some embodiments, the subject receiving the population of CAR-expressing cells contacted ex vivo with the BTK inhibitor (e.g., vecabrutinib) has not received a dose of the BTK inhibitor (e.g., vecabrutinib).

In some embodiments, the subject receiving the population of CAR-expressing cells contacted ex vivo with the BTK inhibitor (e.g., vecabrutinib) has received or will receive a dose of the BTK inhibitor (e.g., vecabrutinib) in addition to the population of CAR-expressing cells. In some embodiments, the subject is administered (i) a population of CAR-expressing cells contacted ex vivo with the BTK inhibitor, or pharmaceutical composition thereof, and (ii) a BTK inhibitor described herein (e.g., vecabrutinib), or pharmaceutical composition thereof.

In some aspects, the present disclosure provides a method of ameliorating toxicity after CAR T-cell (CART) therapy for hematological cancers, without significantly impairing anti-tumor effect of the CART therapy, comprising administering to the subject a combination of the present disclosure, or a pharmaceutical composition of the present disclosure.

In some aspects, the present disclosure provides a method of ameliorating cytokine release syndrome (CRS) severity or preventing CRS after CAR T-cell therapy for hematological cancers, without significantly impairing anti-tumor effect of the CART therapy, comprising administering to the subject a combination of the present disclosure, or a pharmaceutical composition of the present disclosure.

In some aspects, the present disclosure provides a method of ameliorating CRS severity after CAR T-cell therapy for hematological cancers, without significantly impairing anti-tumor effect of the CART therapy, comprising administering to the subject a combination of the present disclosure, or a pharmaceutical composition of the present disclosure.

In some aspects, the present disclosure provides a method of ameliorating neurotoxicity (NT) or prevent NT after CAR T-cell therapy for hematological cancers, without significantly impairing anti-tumor effect of the CART therapy, comprising administering to the subject a combination of the present disclosure, or a pharmaceutical composition of the present disclosure.

In some aspects, the present disclosure provides a method of ameliorating NT after CAR T-cell therapy for hematological cancers, without significantly impairing anti-tumor effect of the CART therapy, comprising administering to the subject a combination of the present disclosure, or a pharmaceutical composition of the present disclosure.

In some aspects, the present disclosure provides that a bruton's tyrosine kinase (BTK) inhibitor can ameliorate toxicities after CAR T-cell (CART) therapy for hematological cancers, without significantly impairing anti-tumor effect of the CART therapy.

In some aspects, the present disclosure provides that a BTK inhibitor can ameliorate cytokine release syndrome (CRS) severity or prevent CRS after CAR T-cell therapy for hematological cancers, without significantly impairing anti-tumor effect of the CART therapy.

In some aspects, the present disclosure provides that a BTK inhibitor can ameliorate CRS severity after CAR T-cell therapy for hematological cancers, without significantly impairing anti-tumor effect of the CART therapy.

In some aspects, the present disclosure provides that a BTK inhibitor can ameliorate neurotoxicity (NT) or prevent NT after CAR T-cell therapy for hematological cancers, without significantly impairing anti-tumor effect of the CART therapy.

In some aspects, the present disclosure provides that a BTK inhibitor can ameliorate NT after CAR T-cell therapy for hematological cancers, without significantly impairing anti-tumor effect of the CART therapy.

In some embodiments, (i) and (ii) are administered simultaneously. In some embodiments, (i) and (ii) are administered in separate formulations. In some embodiments, (i) and (ii) are administered in the same formulation. In some embodiments, (i) and (ii) are administered sequentially. In some embodiments, (i) and (ii) are administered in temporal proximity.

### Dosing Regimens

In some embodiments, the CAR-expressing cell and the inhibitor are administered sequentially, concurrently, or within a treatment interval.

In some embodiments, the CAR-expressing cell and the inhibitor are administered sequentially.

In some embodiments, the inhibitor is administered prior to administration of the CAR-expressing cell.

In some embodiments, the inhibitor is administered after the administration of the CAR-expressing cell.

In some embodiments, the inhibitor and CAR-expressing cell are administered simultaneously or concurrently.

In some embodiments, the CAR-expressing cell and the inhibitor are administered in a treatment interval.

In some embodiments, the treatment interval comprises a single dose of the inhibitor and a single dose of the CAR-expressing cell.

In some embodiments, the treatment interval comprises multiple doses of the inhibitor and a single dose of the CAR-expressing cell.

In some embodiments, the treatment interval comprises multiple doses of the inhibitor and multiple doses of the CAR-expressing cell.

In some embodiments, the CAR-expressing cell is CART19.

In some embodiments, the inhibitor is vecabrutinib.

### BTK Inhibitor

In some embodiments, the BTK inhibitor (e.g., vecabrutinib), is administered multiple times in multiple doses. In some embodiments, the BTK inhibitor (e.g., vecabrutinib) is administered once. In some embodiments, the BTK inhibitor (e.g., vecabrutinib), is administered six times daily, five times daily, four times daily, three times daily, twice daily, once daily, every other day, every three days, twice weekly, once weekly, or only one time prior to or subsequently to initiation of administration of the CAR therapy (e.g., CART therapy).

In some embodiments, the BTK inhibitor (e.g., vecabrutinib) is administered in multiple doses in regular intervals prior to, during, during the course of, and/or after the period of administration of the CAR therapy (e.g., CART therapy). In some embodiments, the BTK inhibitor (e.g., vecabrutinib) is administered in one or more doses in regular intervals prior to the administration of the CAR therapy (e.g., CART therapy). In some embodiments, the BTK inhibitor (e.g., vecabrutinib), is administered in one or more doses in regular intervals after the administration of the CAR cell therapy (e.g., CART therapy). In some embodiments, one or more of the doses of the BTK inhibitor (e.g., vecabrutinib), can occur simultaneously with the administration of a dose of the CAR cell therapy (e.g., CART therapy).

In some embodiments, the dose, frequency, duration, timing and/or order of administration of the BTK inhibitor (e.g., vecabrutinib), is determined, based on particular thresholds or criteria of results of the screening step and/or assessment of treatment outcomes.

In some embodiments, the BTK inhibitor (e.g., vecabrutinib), is administered in a dosage amount of from about 0.2 mg per kg body weight of the subject (mg/kg) to about 200 mg/kg, about 0.2 mg/kg to about 100 mg/kg, about 0.2 mg/kg to about 50 mg/kg, about 0.2 mg/kg to about 10 mg/kg, about 0.2 mg/kg to about 1.0 mg/kg, about 1.0 mg/kg to about 200 mg/kg, about 1.0 mg/kg to about 100 mg/kg, about 1.0 mg/kg to about 50 mg/kg, about 1.0 mg/kg to about 10 mg/kg, about 10 mg/kg to about 200 mg/kg, about 10 mg/kg to about 100 mg/kg, about 10 mg/kg to about 50 mg/kg, about 50 mg/kg to about 200 mg/kg, about 50 mg/kg to about 100 mg/kg, or about 100 mg/kg to about 200 mg/kg. In some embodiments, the BTK inhibitor (e.g., vecabrutinib) is administered at a dose of about 0.2 mg per kg body weight of the subject (mg/kg) to about 50 mg/kg, about 0.2 mg/kg to about 25 mg/kg, about 0.2 mg/kg to about 10 mg/kg, about 0.2 mg/kg to about 5 mg/kg, about 0.2 mg/kg to about 1.0 mg/kg, about 1.0 mg/kg to about 50 mg/kg, about 1.0 mg/kg to about 25 mg/kg, about 1.0 mg/kg to about 10 mg/kg, about 1.0 mg/kg to about 5 mg/kg, about 5 mg/kg to about 50 mg/kg, about 5 mg/kg to about 25 mg/kg, about 5 mg/kg to about 10 mg/kg, or about 10 mg/kg to about 25 mg/kg.

In some embodiments, the BTK inhibitor (e.g., vecabrutinib), is administered in a dosage amount of from about 25 mg to about 2000 mg, about 25 mg to about 1000 mg, about 25 mg to about 500 mg, about 25 mg to about 200 mg, about 25 mg to about 100 mg, about 25 mg to about 50 mg, about 50 mg to about 2000 mg, about 50 mg to about 1000 mg, about 50 mg to about 500 mg, about 50 mg to about 200 mg, about 50 mg to about 100 mg, about 100 mg to about 2000 mg, about 100 mg to about 1000 mg, about 100 mg to about 500 mg, about 100 mg to about 200 mg, about 200 mg to about 2000 mg, about 200 mg to about 1000 mg, about 200 mg to about 500 mg, about 500 mg to about 2000 mg, about 500 mg to about 1000 mg, or about 1000 mg to about 2000 mg.

In some embodiments, the BTK inhibitor (e.g., vecabrutinib), is administered at a total daily dosage amount of at least about 50 mg/day, about 100 mg/day, about 150 mg/day, about 175 mg/day, about 200 mg/day, about 250 mg/day, about 280 mg/day, about 300 mg/day, about 350 mg/day, about 400 mg/day, about 420 mg/day, about 440 mg/day, about 460 mg/day, about 480 mg/day, about 500 mg/day, about 520 mg/day, about 540 mg/day, about 560 mg/day, about 580 mg/day, or about 600 mg/day.

In some embodiments, the BTK inhibitor (e.g., vecabrutinib) is administered at an amount of about 500 mg/day. In some embodiments, the BTK inhibitor (e.g., vecabrutinib) is administered at an amount that is less than about 500 mg/day and at least about 25 mg/day. In some embodiments, the BTK inhibitor (e.g., vecabrutinib) is administered at an amount that is less than about 500 mg/day and at least about 50 mg/day. In some embodiments, the BTK inhibitor (e.g., vecabrutinib) is administered at an amount that is less than about 500 mg/day and at least about 100 mg/day. In some embodiments, the BTK inhibitor (e.g., vecabrutinib) is administered at an amount that is less than about 500 mg/day and at least about 200 mg/day. In some embodiments, the BTK inhibitor (e.g., vecabrutinib) is administered at an amount that is less than 500 mg/day and at least about 300 mg/day.

In some embodiments, the BTK inhibitor (e.g., vecabrutinib) is administered once daily. In some embodiments, the BTK inhibitor (e.g., vecabrutinib) is administered twice daily.

In some embodiments, the BTK inhibitor (e.g., vecabrutinib) is administered orally.

In some embodiments, vecabrutinib is administered in a dosage amount of from about 0.2 mg per kg body weight of the subject (mg/kg) to about 200 mg/kg, about 0.2 mg/kg to about 100 mg/kg, about 0.2 mg/kg to about 50 mg/kg, about 0.2 mg/kg to about 10 mg/kg, about 0.2 mg/kg to about 1.0 mg/kg, about 1.0 mg/kg to about 200 mg/kg, about 1.0 mg/kg to about 100 mg/kg, about 1.0 mg/kg to about 50 mg/kg, about 1.0 mg/kg to about 10 mg/kg, about 10 mg/kg to about 200 mg/kg, about 10 mg/kg to about 100 mg/kg, about 10 mg/kg to about 50 mg/kg, about 50 mg/kg to about 200 mg/kg, about 50 mg/kg to about 100 mg/kg, or about 100 mg/kg to about 200 mg/kg. In some embodiments, vecabrutinib is administered at a dose of about 0.2 mg per kg body weight of the subject (mg/kg) to about 50 mg/kg, about 0.2 mg/kg to about 25 mg/kg, about 0.2 mg/kg to about 10 mg/kg, about 0.2 mg/kg to about 5 mg/kg, about 0.2 mg/kg to about 1.0 mg/kg, about 1.0 mg/kg to about 50 mg/kg, about 1.0 mg/kg to about 25 mg/kg, about 1.0 mg/kg to about 10 mg/kg, about 1.0 mg/kg to about 5 mg/kg, about 5 mg/kg to about 50 mg/kg, about 5 mg/kg to about 25 mg/kg, about 5 mg/kg to about 10 mg/kg, or about 10 mg/kg to about 25 mg/kg.

In some embodiments, vecabrutinib is administered in a dosage amount of from about 25 mg to about 2000 mg, about 25 mg to about 1000 mg, about 25 mg to about 500 mg, about 25 mg to about 200 mg, about 25 mg to about 100 mg, about 25 mg to about 50 mg, about 50 mg to about 2000 mg, about 50 mg to about 1000 mg, about 50 mg to about 500 mg, about 50 mg to about 200 mg, about 50 mg to about 100 mg, about 100 mg to about 2000 mg, about 100 mg to about 1000 mg, about 100 mg to about 500 mg, about 100 mg to about 200 mg, about 200 mg to about 2000 mg, about 200 mg to about 1000 mg, about 200 mg to about 500 mg, about 500 mg to about 2000 mg, about 500 mg to about 1000 mg, or about 1000 mg to about 2000 mg.

In some embodiments, vecabrutinib is administered at a total daily dosage amount of at least about 50 mg/day, about 100 mg/day, about 150 mg/day, about 175 mg/day, about 200 mg/day, about 250 mg/day, about 280 mg/day, about 300 mg/day, about 350 mg/day, about 400 mg/day, about 420 mg/day, about 440 mg/day, about 460 mg/day, about 480 mg/day, about 500 mg/day, about 520 mg/day, about 540 mg/day, about 560 mg/day, about 580 mg/day, or about 600 mg/day.

In some embodiments, vecabrutinib is administered at an amount of about 500 mg/day. In some embodiments, vecabrutinib is administered at an amount that is less than about 500 mg/day and at least about 25 mg/day. In some embodiments, vecabrutinib is administered at an amount that is less than about 500 mg/day and at least about 50 mg/day. In some embodiments, vecabrutinib is administered at an amount that is less than about 500 mg/day and at least about 100 mg/day. In some embodiments, vecabrutinib is administered at an amount that is less than about 500 mg/day and at least about 200 mg/day. In some embodiments, vecabrutinib is administered at an amount that is less than 500 mg/day and at least about 300 mg/day.

In some embodiments, vecabrutinib is administered once daily. In some embodiments, vecabrutinib is administered twice daily.

In some embodiments, vecabrutinib is administered orally.

### CAR-Expressing Cells

In some embodiments, one or more doses of CAR-expressing cells (e.g., CART cells) is administered to a subject as part of the combination therapy described herein. In some embodiments, the size or timing of the doses is determined as a function of the particular disease or condition in the subject. It is within the level of a skilled artisan to empirically determine the size or timing of the doses for a particular disease in view of the provided description.

In some embodiments, a population of CAR-expressing cells (e.g., CART cells) is administered to the subject at a range of about 0.1 million to about 100 billion cells and/or that amount of cells per kilogram of body weight of the subject, such as, e.g., 0.1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight of the subject. Dosages may vary depending on attributes particular to the disease or disorder and/or patient and/or other treatments. In some embodiments, such values refer to numbers of CAR-expressing cells; in other embodiments, they refer to the total number of cells administered.

In some embodiments, the patient is administered multiple doses, and each of the doses or the total dose can be within any of the foregoing values.

In some embodiments, the dose of CAR-expressing cells comprises CAR-expressing T cells. In some embodiments, the dose of CAR-expressing T cells comprises CAR-expressing CD4+ T cells, CAR-expressing CD8+ T cells, or CAR-expressing CD4+ and CAR-expressing CD8+ T cells.

In the context of adoptive cell therapy, administration of a given “dose” of cells encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, such as no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period. Thus, in some aspects, the cells of the dose are administered in a single pharmaceutical composition. In some embodiments, the cells of the dose are administered in a plurality of compositions, collectively containing the cells of the dose.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, administration of the BTK inhibitor (e.g., vecabrutinib) in combination with the CAR-expressing cells (e.g., CART19 cells) substantially or significantly increases the expansion or proliferation of the cells in vivo in a subject, thereby reducing the required dose of the cells administered to the subject to produce a desired outcome (e.g., treatment of the disease or disorder) as compared to the CAR-expressing cells administered in the absence of the BTK inhibitor. In some cases, the methods provided herein enable a lower dose of CAR-expressing cells to be administered, to achieve the same or better efficacy of treatment as the dose in a method in which the cell therapy is administered without administering the BTK inhibitor (e.g., vecabrutinib).

### Kits

The present disclosure provides kits for carrying out the methods described herein. In some embodiments, the kit comprises a BTK inhibitor described herein (e.g., vecabrutinib) or a pharmaceutical composition thereof.

Any kit described above can further comprise one or more additional reagents, where such additional reagents are selected from a buffer, a buffer for introducing a BTK inhibitor and/or a polynucleotide (e.g., vector described herein) into a cell, a wash buffer, a control reagent, a control vector, a control polynucleotide, a reagent for in vitro production of a polypeptide encoded by a vector described herein, adaptors for sequencing and the like. A buffer can be a stabilization buffer, a reconstituting buffer, a diluting buffer, or the like.

In some embodiments, the kit comprises a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition (e.g., cancer).

In some embodiments, the kit comprises a container comprising a composition comprising a BTK inhibitor described herein (e.g., vecabrutinib) and a package insert indicating that the composition can be used to treat a particular condition (e.g., cancer) in a subject receiving one or more components of an immunotherapy described herein. In some embodiments, the kit further comprises another or the same container comprising a pharmaceutically acceptable carrier. In some embodiments, the kit further comprises materials such as other buffers, diluents, filters, needles, and/or syringes.

### EXEMPLARY EMBODIMENTS

Embodiment 1. In some aspects, the present disclosure provides use of immune effector cells engineered to express a Chimeric Antigen Receptor (CAR), in combination with a kinase inhibitor, for the treatment of a disease or disorder in a subject in need thereof.

Embodiment 2. In some aspects, the present disclosure provides a combination comprising:
a. immune effector cells engineered to express a Chimeric Antigen Receptor (CAR); and
b. a BTK inhibitor.

Embodiment 3. In some aspects, the present disclosure provides a method of treating or preventing a disease or disorder in a subject in need thereof, comprising administering to the subject a therapeutically effective amount of the combination of Embodiment 2.

Embodiment 4. In some aspects, the present disclosure provides a combination of Embodiment 2 for use in treating or preventing a disease or disorder.

Embodiment 5. In some aspects, the present disclosure provides use of the combination of Embodiment 2 in the manufacture of a medicament for treating or preventing a disease or disorder.

Embodiment 6. In some aspects, the present disclosure provides the method, use, or combination for use of any one of Embodiments 2-5, wherein the immune effector cells are T-cells.

Embodiment 7. In some aspects, the present disclosure provides the method, use, or combination for use of Embodiment 6, wherein the T-cells are CD19 CAR T-cells.

Embodiment 8. In some aspects, the present disclosure provides the method, use, or combination for use of any one of Embodiments 2-7, wherein the kinase inhibitor is a BTK inhibitor.

Embodiment 9. In some aspects, the present disclosure provides the method, use, or combination for use of any one of Embodiments 2-8, wherein the BTK inhibitor is a reversible BTK inhibitor.

Embodiment 10. In some aspects, the present disclosure provides the method, use, or combination for use of Embodiment 8 or Embodiment 9, wherein the BTK inhibitor is vecabrutinib.

Embodiment 11. In some aspects, the present disclosure provides the method, use, or combination for use of any one of Embodiments 1-10, wherein the disease or disorder is a hematological malignancy.

Embodiment 12. In some aspects, the present disclosure provides the method, use, or combination for use of any one of Embodiments 1-11, wherein the BTK inhibitor ameliorates toxicities after CAR T-cell (CART) therapy.

Embodiment 13. In some aspects, the present disclosure provides the method, use, or combination for use of any one of Embodiments 1-12, wherein the BTK inhibitor ameliorates cytokine release syndrome (CRS) severity or prevents CRS after CAR T-cell therapy.

Embodiment 14. In some aspects, the present disclosure provides the method, use, or combination for use of any one of Embodiments 1-12, wherein the BTK inhibitor ameliorates neurotoxicity (NT) or prevents NT after CAR T-cell therapy.

Embodiment 15. In some aspects, the present disclosure provides the method, use, or combination for use of any one of Embodiments 1-14, wherein the BTK inhibitor does not significantly impair anti-tumor effect of the CART therapy.

Embodiment 16. In some aspects, the present disclosure provides the method, use, or combination for use of any one of Embodiments 1-15, wherein the BTK inhibitor increases in vivo proliferation of CART cells.

Embodiment 17. In some aspects, the present disclosure provides the method, use, or combination for use of any one of Embodiments 1-15, wherein the BTK inhibitor increases in vivo expansion of CART cells.

Embodiment 18. In some aspects, the present disclosure provides the method, use, or combination for use of any one of Embodiments 1-15, wherein the BTK inhibitor reduces in vivo immune suppression of CART cells.

Embodiment 19. In some aspects, the present disclosure provides a method for treating or delaying progression of a cancer in a subject comprising administering to the subject a therapeutically effective amount of a reversible BTK inhibitor, wherein the subject is receiving or has received a CART cell therapy that specifically targets an antigen associated with or expressed by a plurality of cancer cells.

Embodiment 20. In some aspects, the present disclosure provides a method of inducing or promoting an anti-tumor immune response in a subject with a cancer, comprising administering to the subject a therapeutically effective amount of a reversible BTK inhibitor, wherein the subject is receiving or has received a CART cell therapy that specifically targets an antigen associated with or expressed by a plurality of cancer cells.

Embodiment 21. In some aspects, the present disclosure provides a method of increasing activation, proliferation, and/or expansion of CART cells in a subject having a cancer, comprising administering to the subject a therapeutically effective amount of a reversible BTK inhibitor, wherein the subject is receiving or has received a CART cell therapy that specifically targets an antigen associated with or expressed by a plurality of cancer cells.

Embodiment 22. In some aspects, the present disclosure provides the method of any one of Embodiments 19-21, wherein the subject is administered the reversible BTK inhibitor prior to receiving the CART cell therapy.

Embodiment 23. In some aspects, the present disclosure provides the method of any one of Embodiments 19-21, wherein the subject is administered the reversible BTK inhibitor after receiving the CART cell therapy.

Embodiment 24. In some aspects, the present disclosure provides the method of any one of Embodiments 19-21, wherein the reversible BTK inhibitor and CART cell therapy are administered simultaneously.

Embodiment 25. In some aspects, the present disclosure provides the method of any one of Embodiments 19-24, wherein the cancer is a hematological malignancy.

Embodiment 26. In some aspects, the present disclosure provides the method of Embodiment 25, wherein the hematological malignancy comprises a B cell malignancy.

Embodiment 27. In some aspects, the present disclosure provides the method of Embodiment 26, wherein the B cell malignancy is a leukemia, a lymphoma, or a myeloma.

Embodiment 28. In some aspects, the present disclosure provides the method of Embodiment 26 or Embodiment 27, wherein the B cell malignancy is non-Hodgkin lymphoma (NHL), acute lymphoblastic leukemia (ALL), chronic lymphocytic leukemia (CLL), small lymphocytic lymphoma (SLL), diffuse large B-cell lymphoma (DLBCL), lymphoplasmacytoid lymphoma (LL), Waldenstrom macroglobulinemia (MG), mantle-cell lymphoma (MCL), follicular lymphoma (FL), marginal zone lymphoma (MZL), acute myeloid leukemia (AML), or multiple myeloma.

Embodiment 29. In some aspects, the present disclosure provides the method of any one of Embodiments 19-28, wherein the antigen is a B cell antigen.

Embodiment 30. In some aspects, the present disclosure provides the method of Embodiment 29, wherein the B cell antigen is selected from CD10, CD19, CD20, CD22, CD34, CD123, FLT-3, ROR1, CD79b, CD179b, and CD79a.

Embodiment 31. In some aspects, the present disclosure provides the method of Embodiment 29 or Embodiment 30, wherein the B cell antigen is CD19.

Embodiment 32. In some aspects, the present disclosure provides the method of Embodiment 31, wherein the CART cell therapy is selected from Kymriah™, Tecartus™, Breyanzi™, and Yescarta™.

Embodiment 33. In some aspects, the present disclosure provides the method of any one of Embodiments 19-32, wherein the subject is human.

Embodiment 34. In some aspects, the present disclosure provides a kit comprising a container comprising a composition comprising a reversible BTK inhibitor, and optionally a pharmaceutically acceptable carrier, and a package insert comprising instructions for administration of the reversible BTK inhibitor for treating or delaying progression of cancer in a subject receiving CART cell therapy.

Embodiment 35. In some aspects, the present disclosure provides a kit comprising a medicament comprising a composition comprising a reversible BTK inhibitor, and optionally a pharmaceutically acceptable carrier, and a package insert comprising instructions for administration of the reversible BTK inhibitor for treating or delaying progression of cancer in a subject receiving CART cell therapy.

Embodiment 36. In some aspects, the present disclosure provides a kit comprising a container comprising a composition comprising a reversible BTK inhibitor, and optionally a pharmaceutically acceptable carrier, and a package insert comprising instructions for administration of the reversible BTK inhibitor for inducing or promoting an anti-tumor immune response in a subject receiving CART cell therapy.

Embodiment 37. In some aspects, the present disclosure provides a kit comprising a medicament comprising a composition comprising a reversible BTK inhibitor, and optionally a pharmaceutically acceptable carrier, and a package insert comprising instructions for administration of the reversible BTK inhibitor for inducing or promoting an anti-tumor immune response in a subject receiving CART cell therapy.

Embodiment 38. In some aspects, the present disclosure provides a kit comprising a container comprising a composition comprising a reversible BTK inhibitor, and optionally a pharmaceutically acceptable carrier, and a package insert comprising instructions for administration of the reversible BTK inhibitor for increasing activation, proliferation, and/or expansion of CART cells in a subject receiving CART cell therapy.

Embodiment 39. In some aspects, the present disclosure provides a kit comprising a medicament comprising a composition comprising a reversible BTK inhibitor, and optionally a pharmaceutically acceptable carrier, and a package insert comprising instructions for administration of the reversible BTK inhibitor for increasing activation, proliferation, and/or expansion of CART cells in a subject receiving CART cell therapy.

Embodiment 40. In some aspects, the present disclosure provides use of a reversible BTK inhibitor for treating or delaying progression of a cancer in a subject receiving or that has received a CART cell therapy.

Embodiment 41. In some aspects, the present disclosure provides use of a reversible BTK inhibitor in the manufacture of a medicament for treating or delaying progression of a cancer in a subject receiving or that has received a CART cell therapy.

Embodiment 42. In some aspects, the present disclosure provides use of a reversible BTK inhibitor for inducing or promoting an anti-tumor immune response in a subject receiving or that has received a CART cell therapy.

Embodiment 43. In some aspects, the present disclosure provides use of a reversible BTK inhibitor in the manufacture of a medicament for inducing or promoting an anti-tumor immune response in a subject receiving or that has received a CART cell therapy.

Embodiment 44. In some aspects, the present disclosure provides use of a reversible BTK inhibitor for increasing activation, proliferation, and/or expansion of CART cells in a subject receiving or has received a CART cell therapy.

Embodiment 45. In some aspects, the present disclosure provides use of a reversible BTK inhibitor in the manufacture of a medicament for increasing activation, proliferation, and/or expansion of CART cells in a subject receiving or has received a CART cell therapy.

Embodiment 46. In some aspects, the present disclosure provides the method of any one of Embodiments 19-33, the kit of any one of Embodiments 34-39, or the use of any one of Embodiments 40-45, wherein the reversible BTK inhibitor selectively inhibits BTK with a half-maximal inhibitory concentration (IC₅₀) of less than about 100 nM, about 90 nM, about 80 nM, about 70 nM, about 60 nM, about 50 nM, about 40 nM, about 30 nM, or about 20 nM.

Embodiment 47. In some aspects, the present disclosure provides the method, kit, or use of Embodiment 46, wherein the reversible BTK inhibitor inhibits ITK.

Embodiment 48. In some aspects, the present disclosure provides the method, kit, or use of Embodiment 47, wherein the reversible BTK inhibitor inhibits ITK with a half-maximal inhibitory concentration (IC₅₀) of less than about 100 nM, about 90 nM, about 80 nM, about 70 nM, about 60 nM, about 50 nM, about 40 nM, about 30 nM, or about 20 nM.

Embodiment 49. In some aspects, the present disclosure provides the method, kit, or use of any one of Embodiments 46-48, wherein the reversible BTK inhibitor inhibits BTK comprising a mutation selected from C481R and C481S.

Embodiment 50. In some aspects, the present disclosure provides the method, kit, or use of any one of Embodiments 46-49, wherein the reversible BTK inhibitor is vecabrutinib.

### EXAMPLES

### Example 1: Effect of Vecabrutinib on CART19-Mediated Killing of CD19-Expressing JeKo-1 Tumor Cells

CART19 cells were generated using a second generation anti-hCD19 CAR (CART19).

To generate the CART19 cells, PBMCs were isolated from de-identified normal donor blood apheresis cones using SepMate tubes (STEMCELL Technologies, Vancouver, Canada). T cells were separated with negative selection magnetic beads using EasySep™ Human T Cell Isolation Kit (STEMCELL Technologies, Vancouver, Canada). Primary cells were cultured in T Cell Medium made with X-Vivo 15 (Lonza, Walkersville, MD, USA) supplemented with 10% human serum albumin (Corning, NY, USA) and 1% Penicillin-Streptomycin-Glutamine (Gibco, Gaithersburg, MD, USA).

CART19 cells were generated through the lentiviral transduction of normal donor T cells. Second generation CART19 constructs were de novo synthesized (IDT) and cloned into a third generation lentivirus under the control of the EF-1 a promotor. The CD19 directed single chain variable region fragment was derived from the clone FMC63. A second generation 4-1BB co-stimulated (FMC63-41BBz) CAR construct was synthesized and used for these experiments. Lentiviral particles were generated through the transient transfection of plasmid into 293T virus producing cells (gift from the Ikeda lab, Mayo Clinic), in the presence of Lipofectamine 3000 (Invitrogen, Carlsbad, CA, USA), VSV-G and packaging plasmids (Addgene, Cambridge, MA, USA). T cells isolated from normal donors were stimulated using Cell Therapy Systems Dynabeads CD3/CD28 (Life Technologies, Oslo, Norway) at a 1:3 ratio and then transduced with lentivirus particles 24 hours after stimulation at a multiplicity of infection (MOI) of 3.0. To determine titers and subsequently MOI, after lentivirus particles were concentrated, titers were determined by transducing 1×105 primary T cells in 100 ul of T cell medium with 50 ul of lentivirus. First, T cells were stimulated with CD3/CD28 beads and then transduced with lentivirus particles 24 hours later. Transduction was performed in triplicates and at serial dilutions. Fresh T cell medium was added one day later. Two days later, cells were harvested, washed twice with PBS, and CAR expression on T cells was determined by flow cytometry. Titers were determined based on the percentage of CAR positive cells (percentage of CAR+ cells×T cell count at transduction x the specific dilution/volume) and expressed as transducing units/mL (TU/mL). Magnetic bead removal was performed on Day 6 and CAR-T cells were harvested and cryopreserved on Day 8 for future experiments. CAR-T cells were thawed and rested in T cell medium 12 hours prior to their use in experiments.

The efficacy of the CART19 cells in the presence of vecabrutinib was evaluated using an in vitro co-culture assay with hCD19⁺luciferase⁺ mantle cell lymphoma JeKo-1 cell line. CART19 cells and JeKo-1 tumor cells were cultured at a 1:1 ratio for 24 hours in the presence of 1 μM or 10 μM vecabrutinib. A control of CART19/JeKo-1 co-culture treated with DMSO vehicle only was used to determine CART19-mediated killing in the absence of vecabrutinib. Control of untransduced (UTD) T cell/JeKo-1 co-culture in the presence of 1 μM or 10 μM vecabrutinib was used to determine any tumor cell killing induced by vecabrutinib in the absence of CART19 cells. The percentage of tumor cell killing was measured using bioluminescence imaging, with samples treated with D-luciferin prior to imaging.

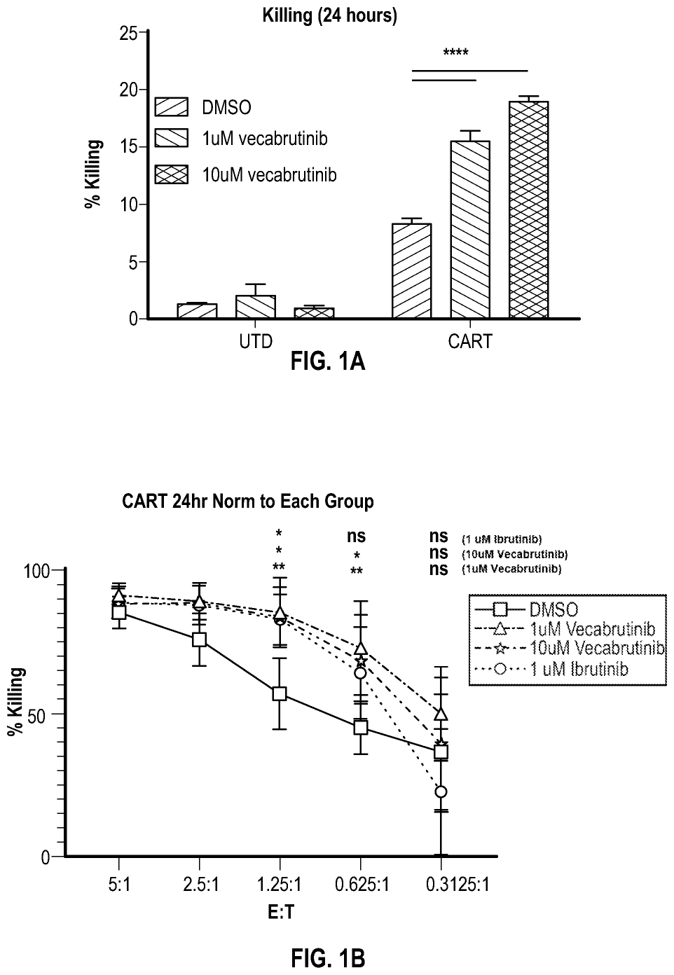

As shown in FIG. 1A, killing of CD19-expressing JeKo-1 tumor cells was significantly increased if CART19 cells were combined with vecabrutinib at either concentration tested.

CAR T19 cell-mediated killing of Jeko-1 tumor cells in the presence of vecabrutinib or ibrutinib was compared. CART19 cells and JeKo-1 tumor cells were co-cultured at a ratio of 5:1, 2.5:1, 1.25:1, 0.625:1, or 0.3125:1 for 24 hours in the presence of 1 μM vecabrutinib, 1 μM ibrutinib, or 10 μM vecabrutinib. Control co-culture was treated with DMSO only. The percentage of tumor cell killing was measured as described above. Additionally, results were normalized to the level of killing measured in tumor cells treated with BTK inhibitor only to account for any cytotoxic effect of the inhibitor on the tumor cells independent of the CART19 cells. As shown in FIG. 1B, vecabrutinib significantly potentiated CART19-mediated killing of Jeko-1 lymphoma cells as compared to ibrutinib when evaluated at the 0.625.1 CART19 to tumor cell ratio.

### Example 2: Effect of Vecabrutinib on CART19 Proliferation In Vitro

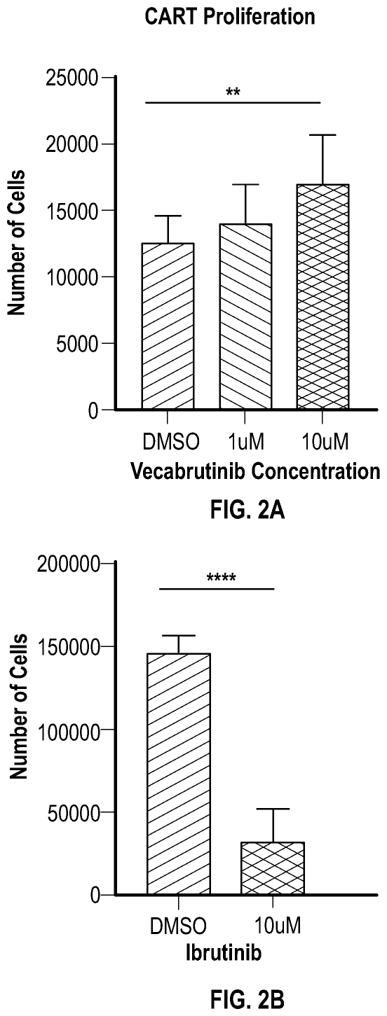

CART19 proliferation in the presence of vecabrutinib was evaluated. CART19 cells were cocultured with irradiated JeKo-1 tumor cells at a 1:1 ratio for 24 hours in the presence of 1 μM or 10 μM vecabrutinib. Control co-culture was treated with DMSO vehicle only. Absolute CD3+ T cell counts were measured by flow cytometry. As shown in FIG. 2A, CART proliferation was comparable in the presence of vecabrutinib compared to the DMSO control. Moreover, CART proliferation increased in the presence of the 10 μM vecabrutinib concentration.

The effect of ibrutinib on proliferation of CART19 cells was also evaluated. CART19 cells were cocultured with irradiated JeKo-1 tumor cells at a 1:1 ratio for 24 hours in the presence of 1 μM or 10 μM. Control co-culture was treated with DMSO only. Absolute CD3+ T cell counts were measured by flow cytometry. Treatment with 1 μM ibrutinib resulted in CART19 proliferation that was comparable to the DMSO control (data not shown). In contrast, as shown in FIG. 2B, treatment with 10 μM ibrutinib resulted in a significant decrease in CART19 proliferation compared to control, indicating cytotoxicity at the higher dose of ibrutinib.

Together, these data indicate that vecabrutinib is well-tolerated at concentrations that are toxic for ibrutinib.

### Example 3: Effect of Vecabrutinib on CART19 Cell Degranulation and Cytokine Production In Vitro

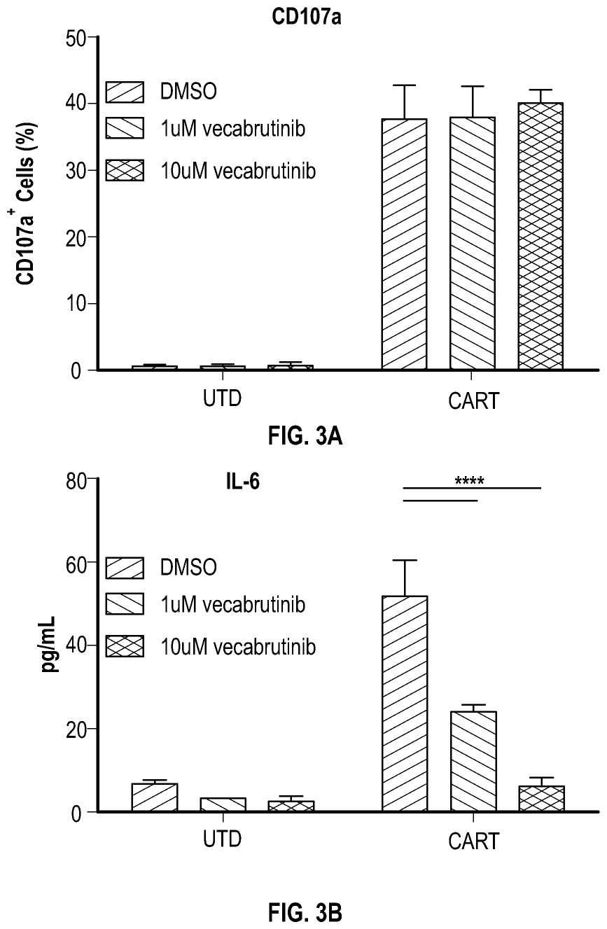

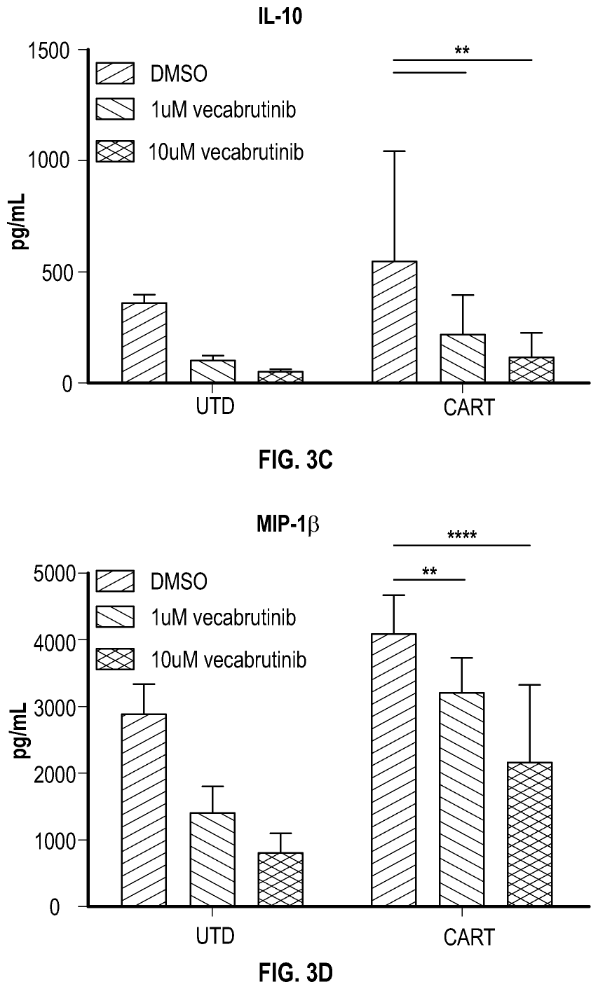

It was evaluated whether treatment with vecabrutinib has an effect on CART19 cell degranulation. Without wishing to be bound by theory, an effect on CART19 cell degranulation may have an important function in CART-mediated killing of target cells. CART19 cells were cocultured with irradiated JeKo-1 tumor cells at a 1:1 ratio for 4 hours in the presence of 1 μM or 10 μM vecabrutinib. CART19/JeKo-1 co-culture was treated with DMSO vehicle only to evaluate CART19 degranulation in the absence of vecabrutinib. Untransduced T cell/JeKo-1 co-culture treated with 1 μM or 10 μM vecabrutinib were used as a negative control as the non-antigen-specific T cells were not expected to undergo degranulation in the presence of JeKo-1 target cells. CD107α was used as a marker to quantify degranulation, as this marker becomes part of the cellular membrane and accessible to surface staining with CD107α-specific antibody in degranulating cells. Following coculture, cells were fixed, permeabilized, attained and analyzed for CD107 levels by flow cytometry. As shown in FIG. 3A, treatment with vecabrutinib did not impair CART19 degranulation.

The effect of vecabrutinib treatment on CART19 cell cytokine production was also evaluated. CART19 cells were cocultured with irradiated JeKo-1 tumor cells at a 1:1 ratio for 3 days in the presence of 1 μM or 10 μM vecabrutinib. CART19/JeKo-1 co-culture treated with DMSO vehicle only was used to evaluate CART19 cell cytokine production in the absence of vecabrutinib. Untransduced T cell/JeKo-1 coculture treated with 1 μM or 10 μM vecabrutinib were used as a negative control as the non-antigen-specific T cells were not expected to undergo activation and cytokine production in the presence of JeKo-1 target cells. Supernatants were harvested and the levels of multiple cytokines and chemokines were analyzed with 38-multiplex (Millipore). As shown in FIGS. 3B-3D, treatment with vecabrutinib resulted in downregulation of IL-6 (FIG. 3B), IL-10 (FIG. 3C), and MIP-1β (FIG. 3D), which are cytokines known to drive the pathology of cytokine release syndrome.

### Example 4: Vecabrutinib Promotes the Anti-Tumor Effects of CART19 Cells In-Vivo

The impact of vecabrutinib on CART19 cell anti-tumor effects was evaluated using a JeKo-1 lymphoma xenograft mouse model. To establish the mouse model, NSG mice were inoculated with 1×10⁶ luciferase⁺ JeKo-1 tumor cells by intravenous injection at day −14. At day −2, mice were imaged by IVIS to confirm tumor engraftment and randomized into treatment groups. Treatment groups received either CART cells with DMSO vehicle or CART cells in combination with 50 mg/kg vecabrutinib. The control group received untransduced T cells with DMSO vehicle. Administration of vecabrutinib or vehicle commenced one day prior to CART19 administration (i.e., day −1), and was given by oral gavage. Untransduced or CART19 were infused by intravenous administration on day 0 at a dose of 1×10⁶ cells per animal. Vecabrutinib and vehicle were administered by oral gavage twice daily over a period of 4 weeks. Mice were imaged weekly by IVIS to assess tumor burden. Blood samples were collected from the mice on a weekly basis and the level of circulating CART19 cells was measured by flow cytometry. Mouse weight was also measured on a weekly basis as a measure of treatment toxicity.

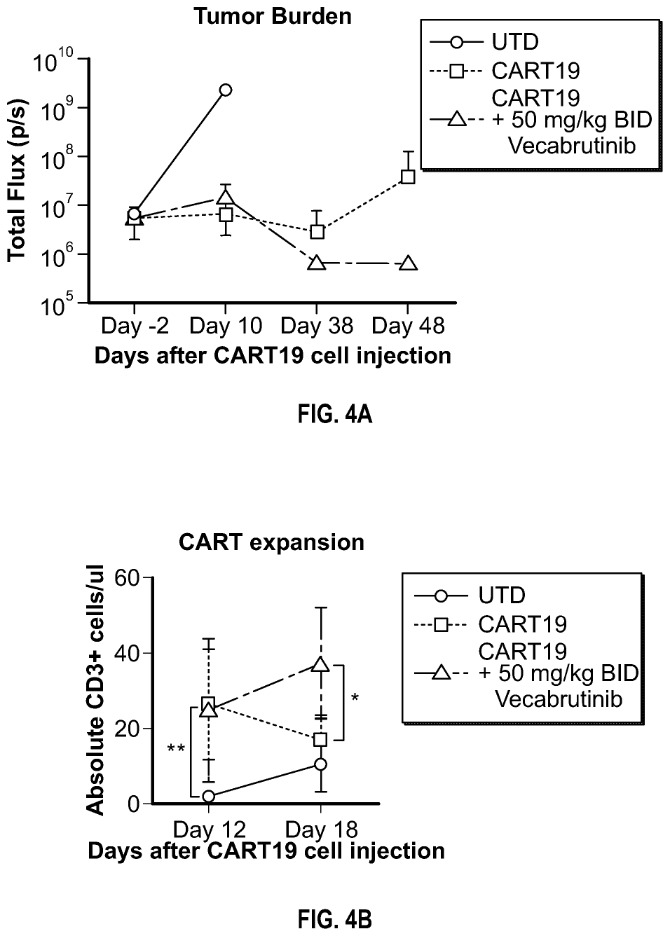

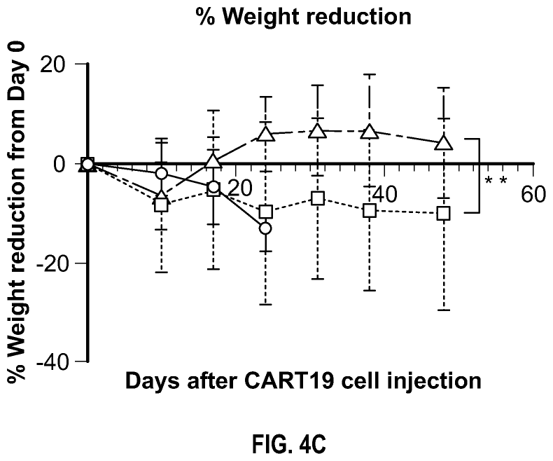

As shown in FIG. 4A, the combination of vecabrutinib and CART19 led to sustained antitumor activity compared to CART19 alone. Moreover, as shown in FIG. 4B, administration of vecabrutinib resulted in increased CART19 proliferation. As shown in FIG. 4C, despite the increased levels of circulating CART19 cells in mice receiving combination of vecabrutinib and CART19, this group experienced weight gain over the treatment course while mice administered CART19 demonstrated weight loss. This indicates that combination of CART19 with vecabrutinib dampens toxicity due to cytokine release syndrome induced by CART19 cells.

### Example 5: Effect of Vecabrutinib on the Transcriptome of CART19 Cells

CART19 cells treated with vecabrutinib in the presence of target cells were evaluated by RNA-sequencing to investigate whether vecabrutinib would alter the transcriptome, e.g., by modulating CART19 cell signaling pathways triggered by CD19-expressing tumor cells. CART19 cells were co-cultured with irradiated JeKo cells at a 1:1 ratio for 72 hours in the presence of 10 μM vecabrutinib. Control cells were co-cultured in the presence of DMSO only. Three biological replicates were used for the vecabrutinib treatment group and the control group. CART19 cells were harvested and mRNA was extracted for library preparation (using a Qiagen miRNeasy micro kit) and next-generation sequencing (NGS) was performed by Illumina. The binary base call data was converted to fastq using Illumina bcl2fastq software. The adapter sequences were removed using Trimmomatic (see, e.g., Bolger, et al. (2014) Bioinformatics btu170), which was confirmed using FastQC. STAR was used for alignment to the human genome annotation GRCh38.p13 (see, e.g., Dobin, et al (2013) Bioinformatics 29:15-21). HTSeq was used to obtain expression counts for each gene (see, e.g., Anders, et al Bioinformatics (2014) 31:166-169). DESeq2 was used to normalize counts across samples, calculate differential expression between groups, and perform multiple hypothesis correction (using the Benjamini-Hochberg procedure) (see, e.g., Love et al (2014) Genome Biology 15:550).

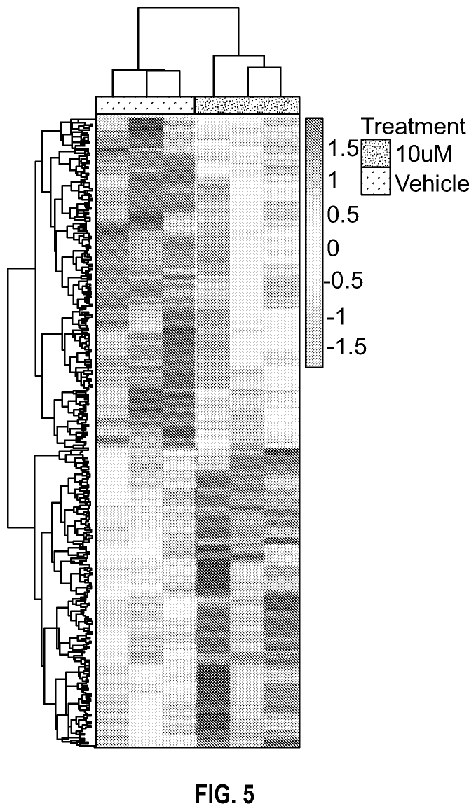

Based on differential expression analysis of CART19 cells treated with 10 μM vecabrutinib and control CART19 cells, approximately 800 genes were found to be differentially regulated as shown in FIG. 5. These included approximately 400 genes that were downregulated and approximately 400 genes that were upregulated with treatment of vecabrutinib. Multiple genes involved in the PI3K/AKT pathway and the Th1 pathway were found to be upregulated in CART19 cells treated with vecabrutinib, indicating activation of these pathways may produce the enhanced CART19 proliferation and target cell cytotoxicity induced in the presence of the inhibitor.

### Example 6: Administration of Vecabrutinib to Human Patients with B Cell Malignancies Results in Reduced Serum Levels of IP-10, MIP-1β, and TNFα

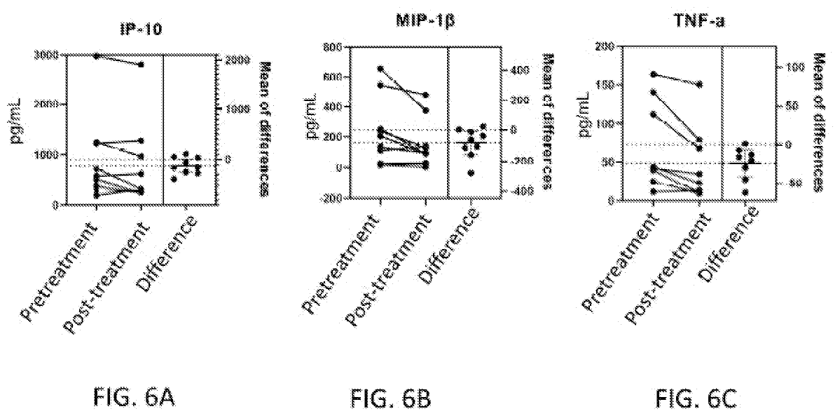

Human cancer patients having B cell malignancies were administered vecabrutinib in a Phase 1 clinical trial and a cytokine panel was used to evaluate cytokine levels in serum at 4 weeks following administration. Briefly, patients in the clinical trial had Chronic Lymphocytic Leukemia (CLL)/Small Lymphocytic Lymphoma (SLL) or Non Hodgkin's lymphoma (NHL) and had failed prior standard of care therapies. NHL indications included lymphoplasmacytoid lymphoma/Waldenstrom's macroglobulinemia (LPL/WM), mantle cell lymphoma (MCL), marginal zone lymphoma (MZL), diffuse large B-cell lymphoma of the activated B-cell subtype (DLBCL-ABC), and follicular lymphoma (FL). A group of 39 patients were orally administered a dose of 20.5 mg, 41 mg, 82 mg, 164 mg, 246 mg, 328 mg, or 410 mg of vecabrutinib as a capsule twice daily. Serum samples were collected from the patients prior to the start of treatment as 4-weeks post-treatment and analyzed by 38-multiplex (Millipore). As shown in FIGS. 6A-6C, oral dosing with vecabrutinib resulted in significantly decreased serum levels of multiple pro-inflammatory cytokines associated with toxicity of CART therapy, including IP-10 (FIG. 6A), MIP-1β (FIG. 6B), and TNFα (FIG. 6C).

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. Use of immune effector cells engineered to express a Chimeric Antigen Receptor (CAR), in combination with a kinase inhibitor, for the treatment of a disease or disorder in a subject in need thereof.

2. A combination comprising:
a. immune effector cells engineered to express a Chimeric Antigen Receptor (CAR); and
b. a BTK inhibitor.

3. A method of treating or preventing a disease or disorder in a subject in need thereof, comprising administering to the subject a therapeutically effective amount of the combination of claim 2.

4. A combination of claim 2 for use in treating or preventing a disease or disorder.

5. Use of the combination of claim 2 in the manufacture of a medicament for treating or preventing a disease or disorder.

6. The method, use, or combination for use of any one of claims 2-5, wherein the immune effector cells are T-cells.

7. The method, use, or combination for use of claim 6, wherein the T-cells are CD19 CAR T-cells.

8. The method, use, or combination for use of any one of claims 2-7, wherein the kinase inhibitor is a BTK inhibitor.

9. The method, use, or combination for use of any one of claims 2-8, wherein the BTK inhibitor is a reversible BTK inhibitor.

10. The method, use, or combination for use of claim 8 or claim 9, wherein the BTK inhibitor is vecabrutinib.

11. The method, use, or combination for use of any one of claims 1-10, wherein the disease or disorder is a hematological malignancy.

12. The method, use, or combination for use of any one of claims 1-11, wherein the BTK inhibitor ameliorates toxicities after CAR T-cell (CART) therapy.

13. The method, use, or combination for use of any one of claims 1-12, wherein the BTK inhibitor ameliorates cytokine release syndrome (CRS) severity or prevents CRS after CAR T-cell therapy.

14. The method, use, or combination for use of any one of claims 1-12, wherein the BTK inhibitor ameliorates neurotoxicity (NT) or prevents NT after CAR T-cell therapy.

15. The method, use, or combination for use of any one of claims 1-14, wherein the BTK inhibitor does not significantly impair anti-tumor effect of the CART therapy.

16. The method, use, or combination for use of any one of claims 1-15, wherein the BTK inhibitor increases in vivo proliferation of CART cells.

17. The method, use, or combination for use of any one of claims 1-15, wherein the BTK inhibitor increases in vivo expansion of CART cells.

18. The method, use, or combination for use of any one of claims 1-15, wherein the BTK inhibitor reduces in vivo immune suppression of CART cells.

19. A method for treating or delaying progression of a cancer in a subject comprising administering to the subject a therapeutically effective amount of a reversible BTK inhibitor, wherein the subject is receiving or has received a CART cell therapy that specifically targets an antigen associated with or expressed by a plurality of cancer cells.

20. A method of inducing or promoting an anti-tumor immune response in a subject with a cancer, comprising administering to the subject a therapeutically effective amount of a reversible BTK inhibitor, wherein the subject is receiving or has received a CART cell therapy that specifically targets an antigen associated with or expressed by a plurality of cancer cells.

21. A method of increasing activation, proliferation, and/or expansion of CART cells in a subject having a cancer, comprising administering to the subject a therapeutically effective amount of a reversible BTK inhibitor, wherein the subject is receiving or has received a CART cell therapy that specifically targets an antigen associated with or expressed by a plurality of cancer cells.

22. The method of any one of claims 19-21, wherein the subject is administered the reversible BTK inhibitor prior to receiving the CART cell therapy.

23. The method of any one of claims 19-21, wherein the subject is administered the reversible BTK inhibitor after receiving the CART cell therapy.

24. The method of any one of claims 19-21, wherein the reversible BTK inhibitor and CART cell therapy are administered simultaneously.

25. The method of any one of claims 19-24, wherein the cancer is a hematological malignancy.

26. The method of claim 25, wherein the hematological malignancy comprises a B cell malignancy.

27. The method of claim 26, wherein the B cell malignancy is a leukemia, a lymphoma, or a myeloma.

28. The method of claim 26 or claim 27, wherein the B cell malignancy is non-Hodgkin lymphoma (NHL), acute lymphoblastic leukemia (ALL), chronic lymphocytic leukemia (CLL), small lymphocytic lymphoma (SLL), diffuse large B-cell lymphoma (DLBCL), lymphoplasmacytoid lymphoma (LL), Waldenstrom macroglobulinemia (MG), mantle-cell lymphoma (MCL), follicular lymphoma (FL), marginal zone lymphoma (MZL), acute myeloid leukemia (AML), or multiple myeloma.

29. The method of any one of claims 19-28, wherein the antigen is a B cell antigen.

30. The method of claim 29, wherein the B cell antigen is selected from CD10, CD19, CD20, CD22, CD34, CD123, FLT-3, ROR1, CD79b, CD179b, and CD79a.

31. The method of claim 29 or claim 30, wherein the B cell antigen is CD19.

32. The method of claim 31, wherein the CART cell therapy is selected from Kymriah™, Tecartus™, Breyanzi™, and Yescarta™.

33. The method of any one of claims 19-32, wherein the subject is human.

34. A kit comprising a container comprising a composition comprising a reversible BTK inhibitor, and optionally a pharmaceutically acceptable carrier, and a package insert comprising instructions for administration of the reversible BTK inhibitor for treating or delaying progression of cancer in a subject receiving CART cell therapy.

35. A kit comprising a medicament comprising a composition comprising a reversible BTK inhibitor, and optionally a pharmaceutically acceptable carrier, and a package insert comprising instructions for administration of the reversible BTK inhibitor for treating or delaying progression of cancer in a subject receiving CART cell therapy.

36. A kit comprising a container comprising a composition comprising a reversible BTK inhibitor, and optionally a pharmaceutically acceptable carrier, and a package insert comprising instructions for administration of the reversible BTK inhibitor for inducing or promoting an anti-tumor immune response in a subject receiving CART cell therapy.

37. A kit comprising a medicament comprising a composition comprising a reversible BTK inhibitor, and optionally a pharmaceutically acceptable carrier, and a package insert comprising instructions for administration of the reversible BTK inhibitor for inducing or promoting an anti-tumor immune response in a subject receiving CART cell therapy.

38. A kit comprising a container comprising a composition comprising a reversible BTK inhibitor, and optionally a pharmaceutically acceptable carrier, and a package insert comprising instructions for administration of the reversible BTK inhibitor for increasing activation, proliferation, and/or expansion of CART cells in a subject receiving CART cell therapy.

39. A kit comprising a medicament comprising a composition comprising a reversible BTK inhibitor, and optionally a pharmaceutically acceptable carrier, and a package insert comprising instructions for administration of the reversible BTK inhibitor for increasing activation, proliferation, and/or expansion of CART cells in a subject receiving CART cell therapy.

40. Use of a reversible BTK inhibitor for treating or delaying progression of a cancer in a subject receiving or that has received a CART cell therapy.

41. Use of a reversible BTK inhibitor in the manufacture of a medicament for treating or delaying progression of a cancer in a subject receiving or that has received a CART cell therapy.

42. Use of a reversible BTK inhibitor for inducing or promoting an anti-tumor immune response in a subject receiving or that has received a CART cell therapy.

43. Use of a reversible BTK inhibitor in the manufacture of a medicament for inducing or promoting an anti-tumor immune response in a subject receiving or that has received a CART cell therapy.

44. Use of a reversible BTK inhibitor for increasing activation, proliferation, and/or expansion of CART cells in a subject receiving or has received a CART cell therapy.

45. Use of a reversible BTK inhibitor in the manufacture of a medicament for increasing activation, proliferation, and/or expansion of CART cells in a subject receiving or has received a CART cell therapy.

46. The method of any one of claims 19-33, the kit of any one of claims 34-39, or the use of any one of claims 40-45, wherein the reversible BTK inhibitor selectively inhibits BTK with a half-maximal inhibitory concentration (IC₅₀) of less than about 100 nM, about 90 nM, about 80 nM, about 70 nM, about 60 nM, about 50 nM, about 40 nM, about 30 nM, or about 20 nM.

47. The method, kit, or use of claim 46, wherein the reversible BTK inhibitor inhibits ITK.

48. The method, kit, or use of claim 47, wherein the reversible BTK inhibitor inhibits ITK with a half-maximal inhibitory concentration (IC₅₀) of less than about 100 nM, about 90 nM, about 80 nM, about 70 nM, about 60 nM, about 50 nM, about 40 nM, about 30 nM, or about 20 nM.

49. The method, kit, or use of any one of claims 46-48, wherein the reversible BTK inhibitor inhibits BTK comprising a mutation selected from C481R and C481S.

50. The method, kit, or use of any one of claims 46-49, wherein the reversible BTK inhibitor is vecabrutinib.
